(19) United States
(12) Patent Application Publication
(10) Pub. No.: US 2014/0148351 A1
(43) Pub. Date: May 29, 2014

(54) Prediction of Clinical Outcome in Hematological Malignancies Using a Self-Renewal Expression Signature

(71) Applicants: Arash Ash Alizadeh, San Mateo, CA (US); Ravindra Majeti, Stanford, CA (US); Andrew J. Gentles, Menlo Park, CA (US)

(72) Inventors: Arash Ash Alizadeh, San Mateo, CA (US); Ravindra Majeti, Stanford, CA (US); Andrew J. Gentles, Menlo Park, CA (US)

(73) Assignee: THE BOARD OF TRUSTEES OF THE LELAND STANFORD JUNIOR UNIVERSITY, Palo Alto, CA (US)

(21) Appl. No.: 13/825,511

(22) PCT Filed: Sep. 28, 2011
(86) PCT No.: PCT/US11/53718

§ 371 Date: May 24, 2013

Related U.S. Application Data
(60) Provisional application No. 61/404,269, filed on Sep. 30, 2010.

(51) Int. Cl. C12Q 1/68

(52) U.S. Cl. CPC C12Q 1/6886
USPC 506/9

(57) ABSTRACT

Methods, compositions, and kits are provided for providing a diagnosis, a prognosis, or a prediction of responsiveness to a therapy for a patient with a hematological malignancy. In practicing the subject methods, the expression level of at least one leukemia stem cell (LSC) genes in a tissue sample is assayed to obtain an LSC expression representation. The LSC expression representation is then employed to determine if an individual has a hematological malignancy, to provide a prognosis to a patient with a hematological malignancy, and/or to provide a prediction of the responsiveness of a patient with a hematological malignancy to a therapy. Also provided are screening methods for identifying novel therapies for patients with a hematological malignancy, and compositions and kits for use in these screening methods.

### CROSS REFERENCE TO RELATED APPLICATIONS

Pursuant to 35 U.S.C. §119 (e), this application claims priority to the filing date of the U.S. Provisional Patent Application Ser. No. 61/404,269 filed Sep. 30, 2010; the disclosure of which is herein incorporated by reference.

### GOVERNMENT RIGHTS

This invention was made with government support under Grants 1U54CA149145 and U56-CA112973 from the National Cancer Institute. The Government has certain rights in the invention.

### FIELD OF THE INVENTION

This invention pertains to providing a diagnosis, a prognosis, or a prediction of responsiveness to therapy for a patient with a hematological malignancy.

### BACKGROUND OF THE INVENTION

Risk classification and outcome prediction for patients with hematological malignancies have to date been limited to observations of cytogenetic aberrations and gene-specific mutations. However, the current classification system does not fully reflect the molecular heterogeneity of the disease. Thus, there is a need in the art for more risk assessment tools for diagnosing hematological malignancies, providing a prognosis for patients with hematological malignancies, and determining the most appropriate therapy for patients with hematological malignancies. The development of such tools will also contribute to our understanding of the underlying causes of such malignancies and the development of novel treatments. The present invention addresses these issues.

### SUMMARY OF THE INVENTION

Methods, compositions, and kits are provided for providing an evaluation of a patient that may have a hematological malignancy, where that evaluation may be a diagnosis of a hematological malignancy, a prognosis regarding that hematological malignancy, and/or a prediction of responsiveness to a particular therapy for that hematological malignancy. Also provided are screening methods for identifying novel therapies for patients with a hematological malignancy, and compositions and kits for use in these screening methods.

In one aspect of the invention, methods and compositions are provided for diagnosing a patient that may have a hematological malignancy, providing a prognosis to a patient with a hematological malignancy, and/or a prediction of responsiveness to a particular therapy. In performing these methods, a leukemia stem cell (LSC) expression representation for a patient hematologic sample is obtained, wherein the LSC expression representation represents the expression level of one or more, for example two or three, LSC genes selected from the group consisting of CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T. The LSC expression representation is then employed to provide a diagnosis, a prognosis, or determination of a therapeutic treatment for the patient. In some embodiments, the LSC expression representation represents measurements of the expression levels of at least the genes HOPX and GUCY1A3. In some embodiments, the LSC expression representation represents measurements of the expression levels of at least the genes HOPX and IL2RA. In some embodiments, the LSC expression representation represents measurements of the expression levels of at least the genes HOPX, GUCY1A3, and IL2RA. In some embodiments, the LSC expression representation represents measurements of the expression levels of at least the genes CCDC48, FAIM3, GIMAP2, GIMAP7, HSPC159, LOC727893, MMRN1, SLC38A1, VNN1, BIRC3, CD34, EBF₃, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, and TMEM200A. In some embodiments, the LSC expression representation is employed in combination with other clinical methods for patient stratification known in the art to arrive at the diagnosis, prognosis, or prediction.

In some embodiments, the LSC expression representation is an LSC expression profile of the normalized expression level of each of said one or more genes. In some embodiments, the LSC expression representation is an LSC signature, that is, a single metric value that represents the weighted expression levels of the one or more LSC genes in a patient sample, where those weighted expression levels are determined based upon the dataset to which a patient sample belongs. In some embodiments, the LSC expression representation is an LSC score, that is, a single metric value that represents the weighted expression levels of the one or more LSC genes in a patient sample, where those weighted expression levels are determined based upon a reference dataset. In some embodiments, the LSC expression representation is employed by comparing it to the LSC expression representation of one or more reference samples to arrive at a comparison result, which is then used to determine a diagnosis, a prognosis or make a prediction on responsiveness to therapy. In some embodiments, the reference sample is a cell or tissue sample with a known association with a particular risk phenotype.

In some embodiments, the hematological malignancy is a leukemia. In some embodiments, the hematological malignancy is a lymphoma. In some embodiments, the hematological malignancy is a multiple myeloma. In some embodiments, the leukemia is acute myelogenous leukemia (AML). In some embodiments, disease prognosis is a prognosis of overall survival (OS), relapse-free survival (RFS) and/or event-free survival (EFS).

In some aspects of the invention, a kit is provided for use in diagnosing a patient that may have a hematological malignancy, providing a prognosis to a patient with a hematological malignancy, and/or predicting responsiveness to a particular therapy, for example allogeneic hematopoietic stem cell transplantation. In some embodiments, the kit comprises reagents for obtaining an LSC expression representation from a hematologic sample, and an LSC expression representation reference. In some embodiments, the LSC expression representation reference is a sample that can be assayed alongside the patient sample. In some embodiments, LSC expression representation reference is a report of disease diagnosis, disease prognosis, or responsiveness to therapy that is correlated with an LSC expression representation.

In some aspects of the invention, methods are provided for screening a candidate agent for the ability to inhibit a hematological malignancy. In performing these methods, a hematologic sample is contacted with a candidate agent, an LSC expression representation is obtained from the contacted hematologic sample, the LSC expression representation from the contacted hematologic sample is compared to an LSC expression representation from a hematologic sample that has not be contacted with the agent, and the result of the comparison are employed to determine the ability of the candidate agent to inhibit a hematological malignancy.

In some embodiments, the contacting step occurs in vitro. In some embodiments, the contacting step occurs in vivo. In some screening embodiments, the LSC expression representation represents the expression level in the hematologic sample of one or more genes selected from the group consisting of CCDC48, FAIM3, GIMAP2, GIMAP7, HSPC159, ILOC727893, MMRN1, SLC38A1, VNN1, BIRC3, CD34, EBF3, EVI2A, GIMAP6, GUCY1A3, HOPX, ICAM1, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T. In some embodiments, a decrease in the LSC expression representation of one or more genes selected from the group consisting of CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, or TMEM200A indicates that the candidate agent inhibits the hematological malignancy. In some embodiments, the LSC expression representation represents measurements of the expression levels of at least the genes HOPX and GUCY1A3. In some embodiments, the LSC expression representation represents measurements of the expression levels of at least the genes HOPX and IL2RA. In some embodiments, the LSC expression representation represents measurements of the expression levels of at least the genes HOPX, GUCY1A3, and IL2RA. In some embodiments, an increase in the LSC expression representation of one or more genes selected from the group consisting of CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T indicates that the candidate agent inhibits the hematological malignancy.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. An LSC-Enriched Gene Expression Signature is Shared with Normal HSC. (A) Gene expression heatmap, with each column representing the difference in expression between paired LSC/LPC-enriched populations isolated from the same AML patient (n=11 from either Stanford or RIKEN); ‘Hs’ denotes LSC/LPC profile of fractionated primary human patient specimen, and ‘Mm’ represents corresponding samples from murine xenografts. Shown are 52 unique genes differentially expressed between LSC and LPC at 10% FDR by SAM (see also Table 1), with red indicating higher expression in LSC. (B) Enrichment analysis of genes differentially expressed between LSC and LPC (see Table 2 for gene set definitions). All nominal p-values were <0.001. NES: GSEA normalized enrichment score; FDR: false discovery rate. (C) Expression of the LSC signature across AML subpopulations (left) and normal hematopoietic stem and progenitor cell (HSPC) populations involved in myeloid differentiation (right), including AML leukemic stem cell (LSC), leukemic progenitor cell (LPC), and leukemic blast (BLAST) populations, as well as normal hematopoietic stem cell (HSC), multipotent progenitor (MPP), common myeloid progenitor (CMP), granulocyte-monocyte progenitor (GMP), and megakaryocyte-erythrocyte progenitor (MEP). Boxes span the interquartile range, with median depicted by the thick horizontal bar. P-values are for Wilcoxon test comparing LSC to LPC/Blast, and for HSC/MPP compared to CMP/GMP/MEP.

FIG. 2. Kaplan-Meier analysis of the association between the LSC score and survival outcomes in NKAML. Excluding those with APL, patients were dichotomized into high- and low-expression groups according to the median value of the LSC score in the training cohort. Stratification of outcomes using this approach is depicted for OS of NKAML patients in the training set (A), in NKAML from one of the validation sets (Tomasson et al.) for OS (B), and for EFS (C). p-values shown are for the LSC score as a continuous predictor of survival (log-likelihood test; log-rank estimates provided in Table 3). Similar results were obtained in additional independent datasets (FIG. 7 and Table 3). (D) The LSC score was highly associated with initial therapeutic response as determined by the ability to achieve clinical remission in two datasets for which this information was available (Wouters et al. and Wilson et al. p-values derived from t-test; Wouters et al. CR n=62, no CR n=43; Wilson et al. CR n=73, no CR n=60). Boxes indicate the interquartile range, with median shown as the thick horizontal bar. OS=overall survival; EFS=event-free survival; CR=clinical remission.

FIG. 3. Lower Expression of the LSC Score Among Prognostically Favorable Groups. The association of the LSC score with clinical features and known predictors of risk was evaluated in the largest cohort (n=526, Wouters et al.). Expression of the LSC score is depicted in (A) age groups stratified by decade, (B) morphological subtypes per French-American-British (FAB) criteria, and (C) karyotype. APL (FAB M3) and FAB M5 were lower in LSC score than all other FAB subtypes, and also from each other (p<0.001 by Games-Howell test). (D) Evaluation of the LSC score in NKAML stratified by recurrent somatic mutations including FLT3-ITD, NPM1, and CEBPA. In all plots, boxes indicate the interquartile range, with median shown as the thick horizontal bar. For karyotype, asterisks denote groups whose distribution of LSC scores differs from the median across samples. For mutations in NKAML, significant differences are indicated for comparison of cases harboring mutations to the corresponding wild-type.

FIG. 4. Network enrichment of LSC signature genes (IPA). Ingenuity Pathways Analysis (IPA) identified three significant interaction networks involving the genes differentially-expressed between LSC- and LPC-enriched subpopulations. These three networks were components of a larger network, shown here. Red nodes indicate genes up-regulated in LSC vs LPC, while green nodes indicate down-regulated genes.

FIG. 5. HOPX interactions with SOX2, OCT4, NANOG. The IPA network involving HOPX identified direct interactions with the induced pluripotency factors SOX2, NANOG, and OCT4 (Pou5f1); together with the histone deacetylase HDAC2. All direct interactions with HOPX identified by IPA are shown here.

FIG. 6. Cross-validation of LSC model score in the training cohort. 1000 random splits were performed of the training cohort, with the LSC score defined in one half and applied to predict OS in the other half. Shown are the resulting distributions obtained for Cox model z scores, −log(log-likelihood p-value), and hazard ratio (HR). In addition, the scatterplot (bottom-right panel) shows the lower- versus upper-95% confidence intervals of the HR obtained in the 1000 splits.

FIG. 7. Kaplan-Meier analysis of additional patient cohorts. Patients were assigned to LSC-high and LSC-low groups defined by the median LSC score in the training cohort. P-values and hazard ratios are reported in Table 3.

FIG. 8. Comparison of prognostic utility of 10000 randomly generated genesets to the LSC score. From 10000 random selections of genes, only one group performed as well as the LSC score in the training set. However, it did not predict outcome in any of the validation cohorts (unlike the LSC score). Shown are the performances (−log of log-likelihood p-value) of all 10000 random sets in the training set versus one of the validation sets, with the performance of the LSC score highlighted in red. The density of the blue cloud represents the number of random sets occurring in that region of the plot, with singletons occurring in low-density regions shown by black dots.

FIG. 9. LSC score across age group and FAB subtype. Variation of LSC scores in relation to age and FAB for additional cohorts. LSC score is shown by age stratified into decade for (A) Metzeler et al., (B) Tomasson et al., and (C) Wilson et al. Variation by FAB subtype is shown in (D-F) (Metzeler et al., Tomasson et al., Wilson et al.).

FIG. 10. LSC score across karyotype and mutations. Variation of LSC score by karyotype and mutations (in NKAML) for additional cohorts. (A-B) LSC score across karyotypes in Tomasson et al. and Wilson et al. (the dataset of Metzeler et al. contains only NKAML). (C-E) LSC signature in FLT3-ITD wild type, FLT3-ITD mutant, NPM1 wild type and NPM1 mutant for Tomasson et al., Metzeler et al., and Wilson et al.

FIG. 11. LSC signature expression is specific to AML from a particular patient, independent of bone marrow or peripheral blood origin. Clustering of data from bone marrow (BM) and peripheral blood (PB) from five patients shows that the expression pattern of LSC genes is patient-specific independent of the sample origin. Numbers in red indicate the ‘approximately unbiased boostrap probability’ for that branch calculated using the PVclust package in R6.

FIG. 12. Multivariate performance of the three gene model derived in the training set (Metzeler) after genes were normalized to ABL1 to simulate the effect in PCR of normalizing to a housekeeping gene (for which ABL1 is a potential candidate) as described for Table 12, but including cytogenetic risk into the model (for the two datasets that contain samples with cytogenetic abnormalities). See Table 13 for data.

FIG. 13. Performance of high/low LSC score. (A) training set cytogenetic intermediate risk. (B) test set cytogenetic intermediate risk. In both plots, x-axis is survival time in months and y-axis is probability of overall survival.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Methods, compositions, and kits are provided for providing a diagnosis, a prognosis, or a prediction of responsiveness to a therapy for a patient with a hematological malignancy. In practicing the subject methods, the expression level of at least one LSC gene in a tissue sample is assayed to obtain an LSC expression representation. The LSC expression representation is then employed to determine if an individual has a hematological malignancy, to provide a prognosis to a patient with a hematological malignancy, and/or to provide a prediction of the responsiveness of a patient with a hematological malignancy to a therapy. Also provided are screening methods for identifying novel therapies for patients with a hematological malignancy, and compositions and kits for use in these screening methods. These and other objects, advantages, and features of the invention will become apparent to those persons skilled in the art upon reading the details of the compositions and methods as more fully described below.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc. The terms “hematological malignancy”, “hematological tumor”, and “hematological cancer” are used interchangeably and in the broadest sense herein and refer to all stages and all forms of cancer arising from cells of the hematopoietic system.

“Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), prediction of a subject's responsiveness to treatment for the disease or disorder (e.g., positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy).

The term “gene product” or “expression product” are used herein to refer to the RNA transcription products (transcripts) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The term “RNA transcript” as used herein refers to the RNA transcription products of a gene, including, for example, mRNA, an unspliced RNA, a splice variant mRNA, a microRNA, and a fragmented RNA.

The term “expression level” as used herein and as it is applied to a gene refers to the level of a gene product, e.g. the normalized value determined for the RNA expression level of a gene or for the expression level of a polypeptide encoded by the gene.

As used herein, an “LSC gene” is a gene that is specifically expressed in an enriched population of leukemic stem cells (LSC), for example which stem cells may be characterized in AML as Lin-CD34+CD38−, relative to non-LSC populations, e.g. leukemic precursor cell (LPC), which precursor may be characterized in AML as Lin-CD34+CD38+, or leukemic blast cells, which blast cells may be characterized in AML as Lin-CD34−. Unless indicated otherwise, each gene name used herein corresponds to the Official Symbol assigned to the gene and provided by Entrez Gene (URL: www.ncbi.nlm.nih.gov/sites/entrez) as of the filing date of this application. LSC genes are examples of genes that are both prognostic and predictive.

An “LSC expression representation” is a representation of the expression levels of one or more LSC genes. LSC expression representations may be in the form of LSC expression profiles, LSC signatures, or LSC scores.

An “LSC expression profile” is the normalized expression level of one or more LSC genes in a patient sample. Normalization of the expression levels of each of the one or more LSC genes may be by any well-understood method in the art, e.g. by comparison to the expression of a selected housekeeping gene, by comparison to the signal across a whole microarray, etc.

An “LSC signature” and an “LSC score” are each a single metric value that represents the sum of the weighted expression levels of one or more LSC genes in a patient sample. Weighted expression levels are calculated by multiplying the normalized expression level of each gene by its “weight”. For an “LSC signature”, the weight of each gene is determined by analysis of the dataset under study, e.g. by Principle Component Analysis (PCA). In other words, the weight is intrinsic to the dataset. Thus, in such instances as when PCA is used, the LSC signature is the first principle component of the LSC genes in a sample based upon the dataset from which that sample was obtained. For an “LSC score”, the weight is determined by analysis of a reference dataset, or “training set”, e.g. a dataset such as the Metzeler data set in Example 1 below, e.g. by PCA, e.g. as with the weights provided in Table 10. Thus, in such instance as when PCA is used, the LSC score is the weighted sum of expression levels of the LSC genes in a sample, where the weights are defined by their first principal component as defined by a reference dataset.

The term “risk classification” means a level of risk (or likelihood) that a subject will experience a particular clinical outcome. A subject may be classified into a risk group or classified at a level of risk based on the methods of the present disclosure, e.g. high, medium, or low risk. A “risk group” is a group of subjects or individuals with a similar level of risk for a particular clinical outcome.

The term “hazard ratio” means the effect of an explanatory variable on the hazard, or risk, of an event occurring. For example, using a Cox proportional hazards regression model, if a variable, e.g. an LSC score, is prognostic, its hazard rate is different in patients with a particular prognosis relative to the hazard rate of other subclasses, and the hazard ratio of the gene is not equal to 1.

The term “long-term” survival is used herein to refer to survival for a particular time period, e.g., for at least 3 years, more preferably for at least 5 years, taking into consideration the median age at which patients are diagnosed with AML and the median survival of all patients with AML.

The term “Overall Survival” or “OS” is used herein to refer to the time (in years) is measured from diagnosis, study entry, or early randomization (depending on the study design) to death from any cause. OS is defined for all patients of a trial; patients not known to have died at last follow-up are censored on the date at which they were last known to be alive. Overall survival is a term that denotes the chances of staying alive for a group of individuals suffering from a cancer. It denotes the percentage of individuals in the group who are likely to be alive after a particular duration of time.

The term “Relapse-Free Survival” or “RFS”, is used herein to refer to the time (in years) measured from diagnosis, study entry, or early randomization (depending on the study design) to first hematological malignancy recurrence. RFS is defined only for patients achieving complete remission, whether with complete blood count recovery (“CR”, e.g. a blood count comprising less than 5% bone marrow blasts, the absence of blasts with Auer rods, the absence of extramedullary disease, an absolute neutrophil count of greater than 1.0×10⁹/L (1000/μL); a platelet count of greater than 100×10⁹/L (100 000/μL), and an independence from red cell transfusions) or without complete blood count recovery (“CRi”, e.g. complete remission except for residual neutropenia (<1.0×10⁹/L [1000/μL]) or thrombocytopenia (<100×10⁹/L [100 000/μL])). RFS is measured from the date of achievement of a remission until the date of relapse or death from any cause; patients not known to have relapsed or died at last follow-up are censored on the date at which they were last examined.

The term “Event-Free Survival” or “EFS” is used herein to refer to the time (in years) measured from diagnosis, study entry, or early randomization (depending on the study design) to the first subsequent event associated with the disease, e.g. complications from the disease, first malignancy recurrence, or death. EFS is defined for all patients of a trial, and is measured from the date of entry into a study to the date of induction treatment failure, or relapse from CR or CRi, or death from any cause; patients not known to have any of these events are censored on the date they were last examined.

The terms “individual,” “subject,” “host,” and “patient,” are used interchangeably herein and refer to any mammalian subject for whom diagnosis, treatment, or therapy is desired, particularly humans.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

Methods, compositions, and kits are provided for diagnosing a patient with a hematological malignancy, for provided a prognosis to a patient with a hematological malignancy, or for predicting the responsiveness a patient with a hematological malignancy to a therapy. The methods and compositions find use in a variety of applications, including diagnosing a patient with a leukemia, a lymphoma, or a myeloma, providing a patient with a leukemia, a lymphoma, or a myeloma with a prognosis, e.g. overall survival, relapse-free survival, or event-free survival, and providing a prediction of responsiveness of a patient with a leukemia, a lymphoma, or a myeloma to a particular therapy, e.g. allogeneic hematopoietic stem cell transplantation, a chemotherapy, and the like, all of which are useful in guiding clinical decisions regarding the patient.

Obtaining an LSC expression representation. In practicing the subject methods, a leukemia stem cell (LSC) expression representation is obtained for a hematologic sample from a patient. An LSC expression representation is a representation of the expression levels of one or more LSC genes in a sample. An LSC gene is a gene that is specifically expressed in leukemic stem cells (LSC) (Lin-CD34+CD38−) relative to non-LSC populations, e.g. leukemic precursor cells (LPC) (Lin-CD34+CD38+) or leukemic blast cells (Lin-CD34−). Examples of LSC genes include, without limitation, CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T.

To obtain an LSC expression representation, the expression level of at least one LSC gene is measured/determined, i.e. the expression levels of at least 1, 2 or 3 LSC genes is determined, sometimes 4, 5, 6 or 7 genes, sometimes 8-15 LSC genes, sometimes 16-30 LSC genes, sometimes 31-40 LSC genes, sometimes 40-50 LSC genes, sometimes more than 50 LSC genes, e.g. the expression levels of 52, 55, or 60 or more genes is determined. For example, in some embodiments, the expression level of at least one gene, e.g. HOPX (HOP homeobox, the sequence for which may be found at Genbank Accession Nos. NM_{—}032495.5 (isoform a), NM_{—}001145459.1 (isoform b), NM_{—}001145460.1 (isoform c)), or GUCY1A3 (Guanylate cyclase 1, soluble, alpha 3, the sequence for which may be found at Genbank Accession Nos. NM_{—}000856.4 (variant 1), NM_{—}001130682.1 (variant 2), NM_{—}001130683.2 (variant 3), NM_{—}001130684.1 (variant 4), NM_{—}001130685.1 (variant 5), NM_{—}001130686.1 (variant 6), NM_{—}001130687.1 (variant 7)), or IL2RA (interleukin 2 receptor, alpha, the sequence for which may be found at Genbank Accession No. NM_{—}000417.2) may be measured. In some embodiments, the expression level of only one gene is measured, e.g. HOPX, or GUCY1A3. In some embodiments, the expression level of at least two genes may be measured, e.g. of HOPX and GUCY1A3, or of GUCY1A3 and IL2RA, or of HOPX and IL2RA, etc. In some embodiments, the expression level of only two genes is measured, e.g. of HOPX and GUCY1A3, or of GUCY1A3 and IL2RA, or of HOPX and IL2RA, etc. In some embodiments, the expression level of at least three genes may be measured, e.g. HOPX, GUCY1A3, and IL2RA. In some embodiments, the expression level of only three genes is measured, e.g. HOPX, GUCY1A3, and IL2RA. In some embodiments, the expression level of a number of genes may be measured, e.g. the expression of at least CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, and TMEM200A, all of which are upregulated specifically in leukemic stem cells relative to other types of cells in a leukemic tissue sample, or the expression of at least CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T, all of which are differentially expressed specifically in leukemic stem cells relative to other types of cells in a leukemic tissue sample.

An LSC expression representation is obtained by obtaining a hematologic sample, e.g. a sample comprising blood cells, from a subject. Examples of hematologic samples include, without limitation, a peripheral blood sample, a bone marrow sample, a spleen biopsy, a lymph node biopsy, and the like. A sample that is collected may be freshly assayed or it may be stored and assayed at a later time. If the latter, the sample may be stored by any means known in the art to be appropriate in view of the method chosen for assaying LSC gene expression, discussed further below. For example the sample may freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. for a blood sample, to purify leukocytes if, e.g., the gene expression product to be assayed is RNA or intracellular protein, or to purify plasma or serum if, e.g., the gene expression product is a secreted polypeptide. Further fractionation may also be performed, e.g., for a purified leukocyte sample, fractionation by e.g. panning, magnetic bead sorting, or fluorescence activated cell sorting (FACS) may be performed to enrich for particular types of cells, e.g. LSCs, LPCs, blast cells, thereby arriving at an enriched population of LSC, LPC or blast cells for analysis; or, e.g., for a plasma or serum sample, fractionation based upon size, charge, mass, or other physical characteristic may be performed to purify particular secreted polypeptides, e.g. under denaturing or non-denaturing (“native”) conditions, depending on whether or not a non-denatured form is required for detection. One or more fractions are then assayed to measure the expression levels of the one or more LSC genes. In some instances, as when the sample is a tissue biopsy that will be sectioned for analysis, the sample may be embedded in sectioning medium, e.g. OCT or paraffin. The sample is then sectioned, and one or more sections are then assayed to measure the expression levels of the one or more LSC genes.

The expression levels of the one or more LSC genes may be measured by polynucleotide, i.e. mRNA, levels or at protein levels. Exemplary methods known in the art for measuring mRNA expression levels in a sample include hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), PCR-based methods (e.g. reverse transcription PCR(RT-PCR) (Weis et al., Trends in Genetics 8:263-264 (1992)), and antibody-based methods, e.g. immunoassays, e.g., enzyme-linked immunosorbent assays (ELISAs), immunohistochemistry, and flow cytometry (FACS).

For measuring mRNA levels, the starting material is typically total RNA or poly A+ RNA isolated from a suspension of cells, e.g. a peripheral blood sample a bone marrow sample, etc., or from a homogenized tissue, e.g. a homogenized biopsy sample, a homogenized paraffin- or OCT-embedded sample, etc. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g. as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

For measuring protein levels, the amount or level of one or more proteins/polypeptides in the sample is determined, e.g., the protein/polypeptide encoded by the gene of interest. In such cases, any convenient protocol for evaluating protein levels may be employed wherein the level of one or more proteins in the assayed sample is determined.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, western blotting, immunohistochemistry, and flow cytometry. In, for example, flow cytometry methods, the quantitative level of gene products of one or more LSC genes are detected on cells in a cell suspension by lasers. As with ELISAs and immunohistochemistry, antibodies (e.g., monoclonal antibodies) that specifically bind the LSC polypeptides are used in such methods.

The resultant data provides information regarding expression for each of the genes that have been probed, wherein the expression information is in terms of whether or not the gene is expressed and, typically, at what level, and wherein the expression data may be both qualitative and quantitative.

Once the expression level of the one or more LSC genes has been determined, the measurement(s) may be analyzed in any of a number of ways to obtain an LSC expression representation.

For example, an LSC expression representation may be obtained by analyzing the data to generate an expression profile. As used herein, an expression profile is the normalized expression level of one or more LSC genes in a patient sample. An expression profile may be generated by any of a number of methods known in the art. For example, the expression level of each gene may be log₂ transformed and normalized relative to the expression of a selected housekeeping gene, e.g. ABL1, GAPDH, or PGK1, or relative to the signal across a whole microarray, etc. An LSC expression profile is one example of an LSC expression representation.

As another example, an LSC expression representation may be obtained by analyzed the data to generate an LSC signature. An LSC signature is a single metric value that represents the weighted expression levels of the panel of LSC genes assayed in a patient sample, where the weighted expression levels are defined by the dataset from which the patient sample was obtained. An LSC signature for a patient sample may be calculated by any of a number of methods known in the art for calculating gene signatures. For example, the expression levels of each of the one or more LSC genes in a patient sample may be log₂ transformed and normalized, e.g. as described above for generating an LSC expression profile. The normalized expression levels for each gene is then weighted by multiplying the normalized level to a weighting factor, or “weight”, to arrive at weighted expression levels for each of the one or more genes. The weighted expression levels are then totaled and in some cases averaged to arrive at a single weighted expression level for the one or more LSC genes analyzed. For an LSC signature, the weighting factor, or weight, is usually determined by Principle Component Analysis (PCA) of the dataset from which the sample was obtained. The LSC signature is the first principle component of the LSC genes in a sample in a given dataset.

As another example, an LSC expression representation may be obtained by analyzed the data to generate an LSC score. Like an LSC signature, an LSC score is a single metric value that represents the sum of the weighted expression levels of one or more LSC genes in a patient sample. An LSC score is determined by methods very similar to those described above for an LSC signature, e.g. the expression levels of each of the one or more LSC genes in a patient sample may be log₂ transformed and normalized, e.g. as described above for generating an LSC expression profile; the normalized expression levels for each gene is then weighted by multiplying the normalized level to a weighting factor, or “weight”, to arrive at weighted expression levels for each of the one or more genes; and the weighted expression levels are then totaled and in some cases averaged to arrive at a single weighted expression level for the one or more LSC genes analyzed. However, in contrast to an LSC signature, the weighted expression levels are defined by a reference dataset, or “training dataset”, e.g. by Principle Component Analysis of a reference dataset. Any dataset relating to patients having hematological malignancies may be used as a reference dataset. For example, the weights may be determined based upon any of the datasets provided in the examples section below, e.g. the Metzeler dataset, the Tomasson dataset, the Wilson dataset, the Wouter dataset, or the like. Thus, the LSC score is the first principle component of the LSC genes in a sample as defined by a reference dataset.

As discussed above, LSC expression representations are obtained by analyzing the data to generate an expression profile, an LSC signature, or an LSC score. This analysis may be readily performed by one of ordinary skill in the art by employing a computer-based system, e.g. using any hardware, software and data storage medium as is known in the art, and employing any algorithms convenient for such analysis. See, for non-limiting examples, the algorithms described in the Examples section below.

Employing an LSC Expression Representation to Evaluate a Subject.

The LSC expression representation that is obtained may employed to diagnose a hematological malignancy, to provide a prognosis to a patient with a hematological malignancy, and/or to provide a prediction of the responsiveness of a patient with a hematological malignancy to a therapy. Typically, an LSC expression representation is employed by comparing the LSC expression representation to a reference or control, and using the results of that comparison (a “comparison result”) to determine a diagnosis, prognosis or prediction. The terms “reference” and “control” as used herein mean a standardized gene expression profile, gene signature, or gene score to be used to interpret the LSC expression representation of a given patient and assign a diagnostic, prognostic, and/or responsiveness class thereto. The reference or control is typically an LSC expression profile, LSC signature, or LSC score that is obtained from a cell/tissue with a known association with a particular risk phenotype. For example, as disclosed in greater detail in the examples section below, a high-risk phenotype is associated with samples from certain affected patients. As disclosed in the examples section below, a high risk phenotype is also associated with hematopoietic stem cell phenotype. Thus, the reference may be an LSC expression profile, LSC signature, or LSC score from a leukemia patient sample, or an enriched culture of leukemic stem cells (LSC), hematopoietic stem cells (HSC), or multipotent progenitor cells (MPP). As another example, a low-risk phenotype is associated with sample from certain other affected patients. And is also associated with a non-hematopoietic stem cell phenotype. Thus, the reference may be an LSC expression profile, LSC signature, or LSC score from a non-leukemia patient or a patient in a low-risk group, or an enriched culture of leukemic precursor cells (LPC), leukemic blast cells (blasts), common myeloid progenitors (CMP), granulocyte-monocyte progenitors (GMP), or megakaryocyte-erythrocyte progenitors (MEP). If the LSC expression representation is an LSC expression profile, the reference will typically be an LSC expression profile from a control sample, whereas if the LSC expression representation is an LSC signature, the reference will typically be the LSC signature from a control sample, and if the LSC expression representation is an LSC score, the reference will typically be the LSC score from a control sample.

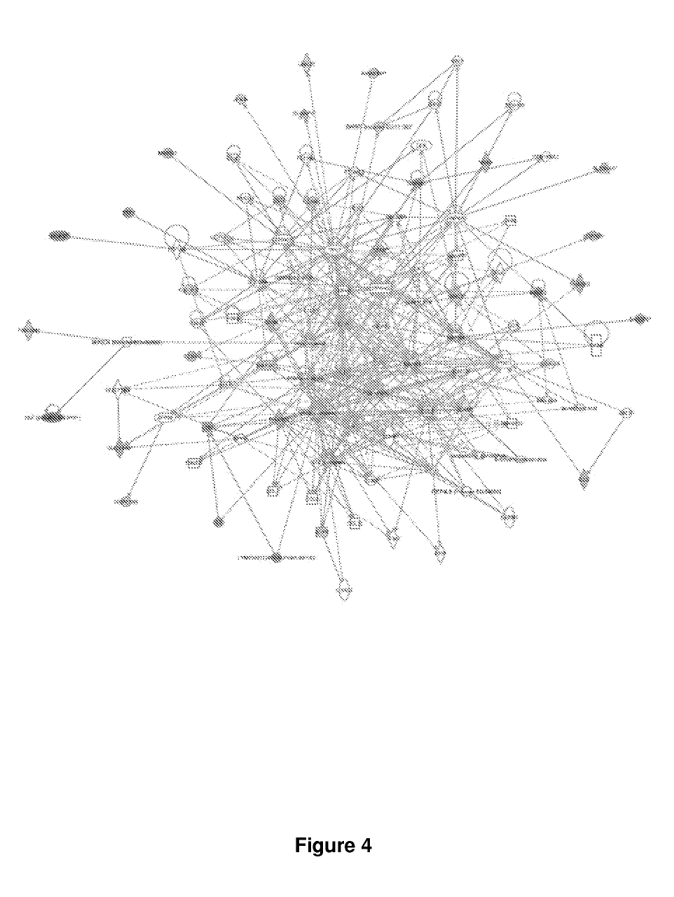

In certain embodiments, the obtained LSC representation is compared to a single reference/control LSC representation to obtain information regarding the phenotype of the tissue being assayed. In yet other embodiments, the obtained LSC representation is compared to two or more different reference/control LSC representations to obtain more in-depth information regarding the phenotype of the assayed tissue. For example, an LSC expression profile may be compared to both a positive LSC expression profiles and a negative LSC expression profiles, and LSC signature may be compared to both a positive LSC signature and a negative LSC signature, or an LSC score may be compared to both a positive LSC score and a negative LSC score to obtain confirmed information regarding whether the tissue has the phenotype of interest. As another example, an LSC signature or score may be compared to multiple LSC signatures or scores, each correlating with a particular diagnosis, prognosis or therapeutic responsiveness, e.g. as might be provided in a report on the correlation between particular LSC signatures/scores and particular disease diagnoses, disease prognoses, or responsiveness to therapy as in, e.g., FIG. 4 of the present disclosure.

As discussed above, an LSC expression representation may be employed to diagnose a hematological malignancy, and if the individual has the hematological malignancy, at what stage that malignancy is at. Examples of hematological malignancies that may be diagnosed using the subject methods include leukemias, lymphomas, and myelomas, including but not limited to Acute myelogenous leukemia (AML), Acute lymphoblastic leukemia (ALL), Chronic myelogenous leukemia (CML), Chronic lymphocytic leukemia (CLL) (called small lymphocytic lymphoma (SLL) when leukemic cells are absent), Acute monocytic leukemia (AMOL), Hodgkin's lymphomas, Non-Hodgkin's lymphomas (e.g. Chronic lymphocytic leukemia (CLL), Diffuse large B-cell lymphoma (DLBCL), Follicular lymphoma (FL), Mantle cell lymphoma (MCL), Marginal zone lymphoma (MZL), Burkitt's lymphoma (BL), Hairy cell leukemia, Post-transplant lymphoproliferative disorder (PTLD), Waldenström's macroglobulinemia/Lymphoplasmacytic lymphoma, Hepatosplenic-T cell lymphoma, and Cutaneous T cell lymphoma (including Sezary's syndrome)), and multiple myeloma. In particular embodiments, the subject methods find utility in diagnosing AML, and further, in diagnosing certain subtypes of AML based on the French-American-British (FAB) criteria. For example, patients with the MO subtype (minimally differentiated acute myeloblastic leukemia) present with a uniquely high LSC signature and LSC score relative to all other FAB subtypes, whereas patients with the M3 subtype (promyelocytic, or acute promyelocytic leukemia (APL)) present with a uniquely low LSC signature and LSC score.

Alternatively or additionally, the LSC expression representation may be employed to provide a prognosis to a patient with one of the aforementioned hematological malignancies. For example, patients can be ascribed to high- or low-risk categories, or high-, intermediate- or low-risk categories, for overall survival, relapse-free survival, event-free survival, etc. depending on whether their LSC signature and/or LSC score is higher or lower than the median score across a cohort of patients with the same disease. An example of this is provided in the examples section below, wherein it is demonstrated by Kaplan-Meier analysis that a high LSC signature and LSC score is negatively correlated with overall survival, relapse-free survival, and event-free survival, and by Kaplan-Meier analysis and risk plot exactly what that prognosis may be.

Alternatively or additionally, the LSC expression representation may be employed to provide a prediction of responsiveness of a patient with one of the aforementioned hematological malignancies to a particular therapy. These predictive methods can be used to assist patients and physicians in making treatment decisions, e.g. in choosing the most appropriate treatment modalities for any particular patient. For example, the LSC expression representation may be used to predict responsiveness to induction chemotherapy, e.g. daunorubicin (DNR), cytarabine (ara-C), idarubicin, thioguanine, etoposide, or mitoxantrone; to antibody therapy, e.g. anti-CD47, anti-CD20, etc., or to stem cell transplantation, e.g. allogenic hematopoietic stem cell transplantation, e.g. from bone marrow. An example of this is provided in the examples section below, wherein it is demonstrated by Kaplan-Meier analysis that a high LSC signature and LSC score is positively correlated with the patient being refractory, i.e. non-responsive, to induction chemotherapy, i.e. the initial chemotherapy treatment. Additionally, the LSC representation may be used on samples collected from patients in a clinical trial and the results of the test used in conjunction with patient outcomes in order to determine whether subgroups of patients are more or less likely to show a response to a new drug than the whole group or other subgroups. Further, such methods can be used to identify from clinical data the subsets of patients who can benefit from therapy. Additionally, a patient is more likely to be included in a clinical trial if the results of the test indicate a higher likelihood that the patient will have a poor clinical outcome if treated with more standardized treatments, and a patient is less likely to be included in a clinical trial if the results of the test indicate a lower likelihood that the patient will have a poor clinical outcome if treated with more standardized treatments.

The subject methods can be used alone or in combination with other clinical methods for patient stratification known in the art, e.g. age, cytogenetics, the presence of certain molecular mutations, the altered expression levels of particular genes, e.g. IL2RA and MSI2, and the like, to provide a diagnosis, a prognosis, or a prediction of responsiveness to therapy. For example, for AML, known clinical prognostic factors associated with favorable outcome include cytogenetic mutations such as t(15;17)PML/RARα, t(8;21)AML1/ETO, 11q23, and inv(16)CBFβ/MYH11, or molecular mutations in FLT3 (e.g., FLT3-ITD, FLT3-D835), NPM1, EVI1, or cEBPα; clinical prognostic factors that have been associated with an intermediate outcome include Normal karyotype, and the cytogenetic mutations +8, +21, +22, del(7q), and del(9q); and clinical prognostic factors that have been associated with an adverse outcome include the cytogenetic mutations del(5q), 11q23, t(6;9), t(9;22), abnormal 3q, complex cytogenetics, and elevated expression levels of IL2Ra and/or MSI2.

In some embodiments, providing an evaluation of a subject for a hematological malignancy, i.e., a diagnosis, a prognosis, or a prediction of responsiveness to therapy, includes generating a written report that includes the artisan's assessment of the subject's current state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject has a hematological malignancy; or a prognosis assessment, i.e. a prediction of the likelihood that a patient with a cancer will have a cancer-attributable death or progression, including recurrence, metastatic spread, and drug resistance; or a treatment assessment, i.e. a prediction as to the likelihood that a cancer patient will have a particular clinical response to treatment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include i) the gene expression levels of one or more LSC genes, ii) the gene expression profiles for one or more LSC genes, and/or iii) an LSC signature and/or LSC score, b) reference values employed, if any; 6) other features.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will have a cancer-attributable death or progression. The interpretive report can include, for example, results of the gene expression analysis, methods used to calculate the LSC expression representation, and interpretation, i.e. prognosis. The assessment portion of the report can optionally also include a Recommendation(s). For example, where the results indicate that the subject will be responsive to induction chemotherapy, the recommendation can include a recommendation that a bone marrow transplant be performed with induction chemotherapy to follow.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Screening Methods

The methods described herein provide a useful system for screening candidate agents for activity in treating a hematological malignancy and the development of drugs for the same. These screening methods are based upon the observation disclosed herein that a high leukemic stem cell (LSC) signature and a high LSC score in a hematologic sample correlates with hematological malignancy and, more particularly, with “high risk” hematological malignancy, i.e. with a hematological malignancy that has a poor outcome for overall survival, relapse-free survival, or event-free survival, and is refractory to induction therapy. Addition of agents that modulate LSC expression representation such that it more closely resembles that of a normal, i.e. non-affected, subject will therefore be useful in treating hematological malignancies.

In screening assays for biologically active agents, cells, usually cultures of cells, e.g. from a subject with a hematological malignancy, are contacted with the candidate agent of interest and the effect of the candidate agent is assessed by monitoring output parameters, such as cell survival, LSC gene expression levels, etc. by methods described above.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

For example, agents can be screened for an activity in modulating LSC gene expression levels. A decrease in the LSC gene expression levels observed, e.g. a 1.5-fold, a 2-fold, a 3-fold or more decrease in the LSC expression profile, LSC signature, or LSC score over that observed in the culture absent the candidate agent would indicate that the candidate agent was an agent that targets LSC cells.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. Various methods can be utilized for quantifying the expression level of LSC genes, as discussed above.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The aforementioned screening assays also find use in determining if a patient with a hematological malignancy will be responsive to a particular therapy. For example, a culture of cells from a hematological tissue sample from the patient is contacted with the therapeutic agent of interest and the effect of the agent is assessed by monitoring output parameters, such as cell survival, LSC gene expression levels, etc. by methods described above. Modulation of the LSC expression representation as discussed above would serve as a useful indicator that the patient is or is not likely to respond to the therapeutic agent.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of assaying gene expression levels, where such reagents may include RNA or protein purification reagents, nucleic acid primers specific for LSC genes, arrays of nucleic acid probes, antibodies to LSC polypeptides (e.g., immobilized on a substrate), signal producing system reagents, etc., depending on the particular detection protocol to be performed. For example, reagents may include PCR primers that are specific for one or more of the LSC genes CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T. Other examples of reagents include arrays that comprise probes that are specific for one or more of the LSC genes, and antibodies to epitopes of the proteins encoded by these LSC genes.

The subject kits may also comprise one or more LSC expression representation references, for use in employing the LSC expression reference obtained from a patient sample. For example, the reference may be a sample of a known phenotype, e.g. an unaffected individual, or an affected individual, e.g. from a particular risk group, that can be assayed alongside the patient sample, or the reference may be a report of disease diagnosis, disease prognosis, or responsiveness to therapy that is known to correlate with one or more LSC expression representations.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Background

A growing body of evidence suggests that specific cancer cell subpopulations possess the ability to initiate and maintain tumors (Jordan C T, et al., Cancer stem cells. N Engl J Med. 2006; 355(12):1253-1261; Reya T, et al. Stem cells, cancer, and cancer stem cells. Nature. 2001; 414(6859):105-111). This model has major implications for the development of novel therapeutic agents (Weissman I. Stem cell research: paths to cancer therapies and regenerative medicine. JAMA. Sep. 21, 2005; 294(11):1359-1366).

Acute myeloid leukemia (AML) is an aggressive clonal malignancy of the bone marrow characterized by the accumulation of early myeloid cells that fail to mature and differentiate. There is significant support that AML is organized as a cellular hierarchy initiated and maintained by self-renewing leukemia stem cells (LSC) that comprise a subset of the total leukemic burden (Jordan C T, et al. supra; Dick J E. Stem cell concepts renew cancer research. Blood. Dec. 15, 2008; 112(13):4793-4807).

AML stem cells were initially identified by prospectively separating primary leukemic specimens into subpopulations based on expression of CD34 and CD38, surface markers that are differentially expressed in the normal hematopoietic hierarchy (Dick, J E, supra). When the function of these subpopulations was assessed by transplantation into immune-deficient mice, leukemia-initiating activity was demonstrated exclusively in the CD34+CD38− fraction (LSC-enriched) (Bonnet D, Dick J E. Human acute myeloid leukemia is organized as a hierarchy that originates from a primitive hematopoietic cell. Nat. Med. July 1997; 3(7):730-737). LSC in turn give rise to CD34+CD38+ leukemia progenitor cells (LPC), which further differentiate into the CD34− leukemic blast population (Dick, J E, supra; Bonnet D. et al., supra).

We define here a gene expression signature of LSC-enriched subpopulations and investigate its clinical relevance using bulk AML data from four independent cohorts representing 1047 adult patients with diverse clinical and pathological features. We define an LSC score and test it for associations with known predictors of risk including cytogenetic subtype, molecularly-defined mutations, and as an independent prognostic factor. We find that higher LSC score is independently predictive of adverse outcomes in all four cohorts, supporting the clinical utility of this model for AML.

### Results

An LSC-Enriched Gene Expression Signature is Shared with Normal HSC.

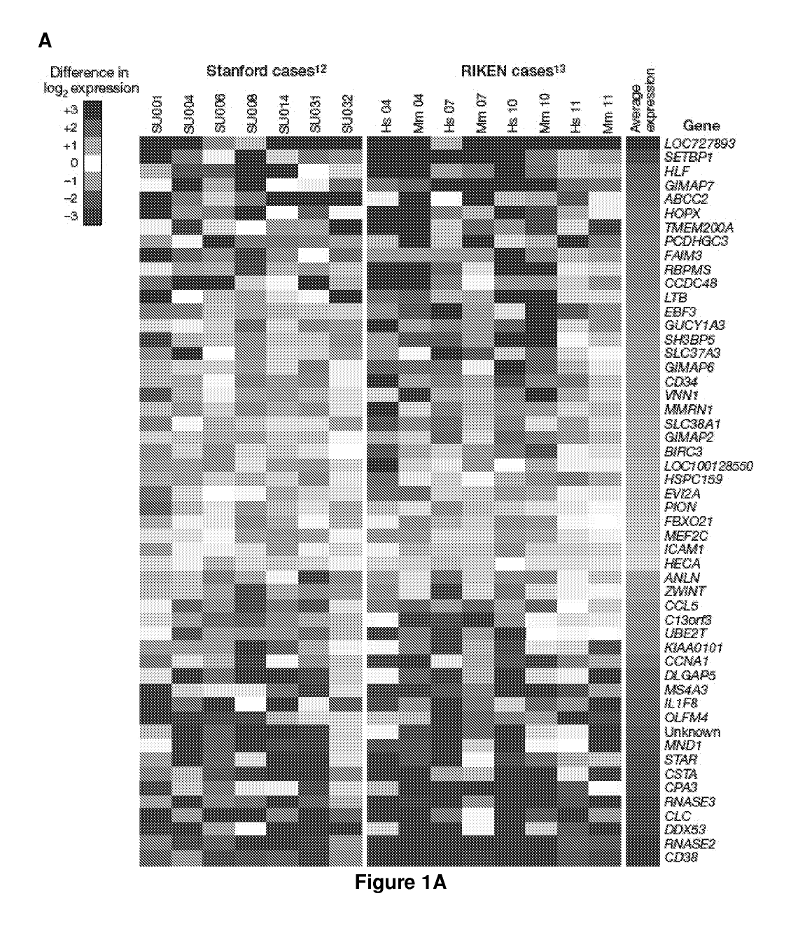

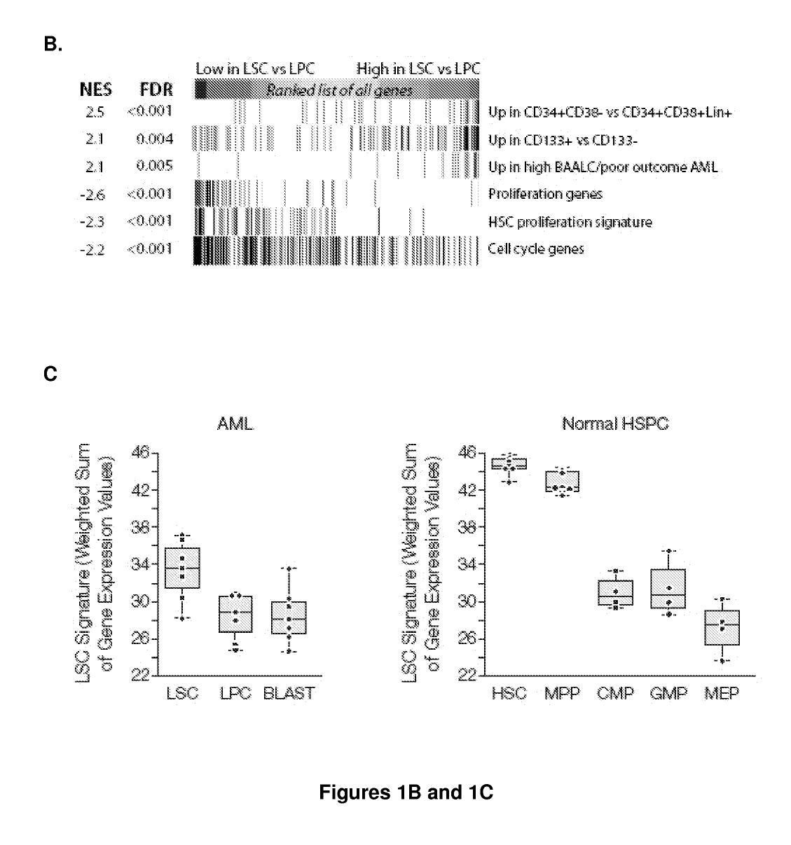

To define a leukemic stem cell signature within AML, we directly compared gene expression profiles of subpopulations with distinct functional capacities for leukemia initiation in transplantation models. Gene expression profiles of enriched LSC and LPC subpopulations were obtained from 7 patients diagnosed with AML at the SUMC (Majeti R, et al. Dysregulated gene expression networks in human acute myelogenous leukemia stem cells. Proc Natl Acad Sci USA. Mar. 3, 2009; 106(9):3396-3401.), and combined with publicly available profiles from 4 additional adult patients with AML, together with their corresponding functionally defined mouse xenografts (Ishikawa F, et al. Chemotherapy-resistant human AML stem cells home to and engraft within the bone-marrow endosteal region. Nat. Biotechnol. November 2007; 25(11):1315-1321). The 15 paired specimens were used to define differentially expressed genes, identifying 21 genes as relatively down-regulated in LSC, and 31 genes as up-regulated (FIG. 1A and Table 1).

Tables 1A and 1B.

Genes differentially expressed between LSC and LPC. Genes distinguishing LSC-enriched from LPC-enriched populations were identified using SAM with paired metric. As described in the results, this approach identified 52 unique genes (see also FIG. 1). (A) 31 genes up-regulated in LSC vs LPC, at 10% false discovery rate. (B) 21 genes down-regulated in LSC vs LPC, at 10% false discovery rate. Tabulated are the Affymetrix probeset name (RefSeq accession followed by _at, as per custom CDF v12), gene name and description, geometric mean fold change (log 2), mean fold change, and FDR.

TABLE 1A
Genes more highly expressed in LSC compared to LPC.

| | | Ave | Mean | FDR |
|---|---|---|---|---|
| Probe | Gene | log2FC | FC | (%) |
| NM_024768_at | CCDC48—Coiled-coil domain containing 48 | 1.53 | 2.89 | 0 |
| NM_001142472_at | FAIM3—Fas apoptotic inhibitory molecule 3 | 1.56 | 2.95 | 0 |
| NM_015660_at | GIMAP2—GTPase, IMAP family member 2 | 0.94 | 1.92 | 0 |
| NM_153236_at | GIMAP7—GTPase, IMAP family member 7 | 1.94 | 3.84 | 0 |
| NM_014181_at | HSPC159—Galectin-related protein | 0.88 | 1.84 | 0 |
| XM_001126245_at | LOC727893—Similar to phosphodiesterase | 2.99 | 7.94 | 0 |
| | 4D interacting protein (myomegalin) | | | |
| NM_007351_at | MMRN1—Multimerin 1 | 1.12 | 2.17 | 0 |
| NM_001077484_at | SLC38A1—Solute carrier family 38, member 1 | 1 | 2 | 0 |
| NM_004666_at | VNN1—Vanin 1 | 1.13 | 2.19 | 0 |
| NM_001165_at | BIRC3—Baculoviral IAP repeat-containing 3 | 0.93 | 1.91 | 7.25 |
| NM_001025109_at | CD34—CD34 molecule | 1.16 | 2.23 | 7.25 |
| NM_001005463_at | EBF3—Early B-cell factor 3 | 1.38 | 2.6 | 7.25 |
| NM_001003927_at | EVI2A—Ecotropic viral integration site 2A | 0.82 | 1.77 | 7.25 |
| NM_024711_at | GIMAP6—GTPase, IMAP family member 6 | 1.17 | 2.25 | 7.25 |
| NM_001130687_at | GUCY1A3—Guanylate cyclase 1, soluble, alpha 3 | 1.34 | 2.53 | 7.25 |
| NM_001145459_at | HOPX—HOP homeobox | 1.7 | 3.25 | 7.25 |
| NM_000201_at | ICAM1—Intercellular adhesion molecule 1 | 0.63 | 1.55 | 7.25 |
| NM_032090_at | PCDHGC3—Protocadherin gamma subfamily C, 3 | 1.56 | 2.95 | 7.25 |
| NM_017439_at | PION—Pigeon homolog (Drosophila) | 0.75 | 1.68 | 7.25 |
| NM_006867_at | RBPMS—RNA binding protein with | 1.54 | 2.91 | 7.25 |
| | multiple splicing | | | |
| NM_015559_at | SETBP1—SET binding protein 1 | 2.25 | 4.76 | 7.25 |
| NM_001018009_at | SH3BP5—SH3-domain binding protein 5 | 1.22 | 2.33 | 7.25 |
| | (BTK-associated) | | | |
| NM_000392_at | ABCC2—ATP-binding cassette, sub- | 1.78 | 3.43 | 8.6 |
| | family C (CFTR/MRP), member 2 | | | |
| NM_015002_at | FBXO21—F-box protein 21 | 0.7 | 1.6 | 8.6 |
| NM_016217_at | HECA—Headcase homolog (Drosophila) | 0.48 | 1.39 | 8.6 |
| NM_002126_at | HLF—Hepatic leukemia factor | 2.03 | 4.08 | 8.6 |
| XM_001716710_at | LOC100128550—Hypothetical protein | 0.88 | 1.84 | 8.6 |
| | LOC100128550 | | | |
| NM_002341_at | LTB—Lymphotoxin beta (TNF | 1.45 | 2.73 | 8.6 |
| | superfamily, member 3) | | | |
| NM_001131005_at | MEF2C—Myocyte enhancer factor 2C | 0.68 | 1.6 | 8.6 |
| NM_032295_at | SLC37A3—Solute carrier family 37 (glycerol- | 1.18 | 2.27 | 8.6 |
| | 3-phosphate transporter), member 3 | | | |
| NM_052913_at | TMEM200A—Transmembrane protein 200A | 1.65 | 3.14 | 8.6 |

TABLE 1B
Genes with lower expression in LSC compared to LPC.

| | | | | |
|---|---|---|---|---|
| NM_001775_at | CD38—CD38 molecule | −3.07 | −8.4 | 0 |
| NM_005213_at | CSTA—Cystatin A (stefin A) | −2.08 | −4.23 | 0 |
| NM_182699_at | DDX53—DEAD (Asp-Glu-Ala-Asp) box | −2.66 | −6.32 | 0 |
| | polypeptide 53 | | | |
| NM_002934_at | RNASE2—Ribonuclease, RNase A | −2.99 | −7.94 | 0 |
| | family, 2 (liver, eosinophil-derived | | | |
| | neurotoxin) | | | |
| NM_002935_at | RNASE3—Ribonuclease, RNase A | −2.47 | −5.54 | 0 |
| | family, 3 (eosinophil cationic protein) | | | |
| NM_001146015_at | ---—--- | −1.91 | −3.76 | 7.25 |
| NM_018685_at | ANLN—Anillin, actin binding protein | −0.93 | −1.91 | 7.25 |
| NM_145061_at | C13orf3—Chromosome 13 open reading | −1.16 | −2.23 | 7.25 |
| | frame 3 | | | |
| NM_002985_at | CCL5—Chemokine (C-C motif) ligand 5 | −1.12 | −2.17 | 7.25 |
| NM_001111047_at | CCNA1—Cyclin A1 | −1.5 | −2.83 | 7.25 |
| NM_001828_at | CLC—Charcot-Leyden crystal protein | −2.47 | −5.54 | 7.25 |
| NM_001870_at | CPA3—Carboxypeptidase A3 (mast cell) | −2.1 | −4.29 | 7.25 |
| NM_014750_at | DLGAP5—Discs, large (Drosophila) | −1.66 | −3.16 | 7.25 |
| | homolog-associated protein 5 | | | |
| NM_014438_at | IL1F8—Interleukin 1 family, member 8 | −1.76 | −3.39 | 7.25 |
| | (eta) | | | |
| NM_014736_at | KIAA0101—KIAA0101 | −1.23 | −2.35 | 7.25 |
| NM_032117_at | MND1—Meiotic nuclear divisions 1 | −1.98 | −3.94 | 7.25 |
| | homolog (S. cerevisiae) | | | |
| NM_001031666_at | MS4A3—Membrane-spanning 4-domains, | −1.68 | −3.2 | 7.25 |
| | subfamily A, member 3 | | | |
| | (hematopoietic cell-specific) | | | |
| NM_006418_at | OLFM4—Olfactomedin | −1.8 | −3.48 | 7.25 |
| NM_000349_at | STAR—Steroidogenic acute regulatory | −2.07 | −4.2 | 7.25 |
| | protein | | | |
| NM_001005413_at | ZWINT—ZW10 interactor | −0.96 | −1.95 | 7.25 |

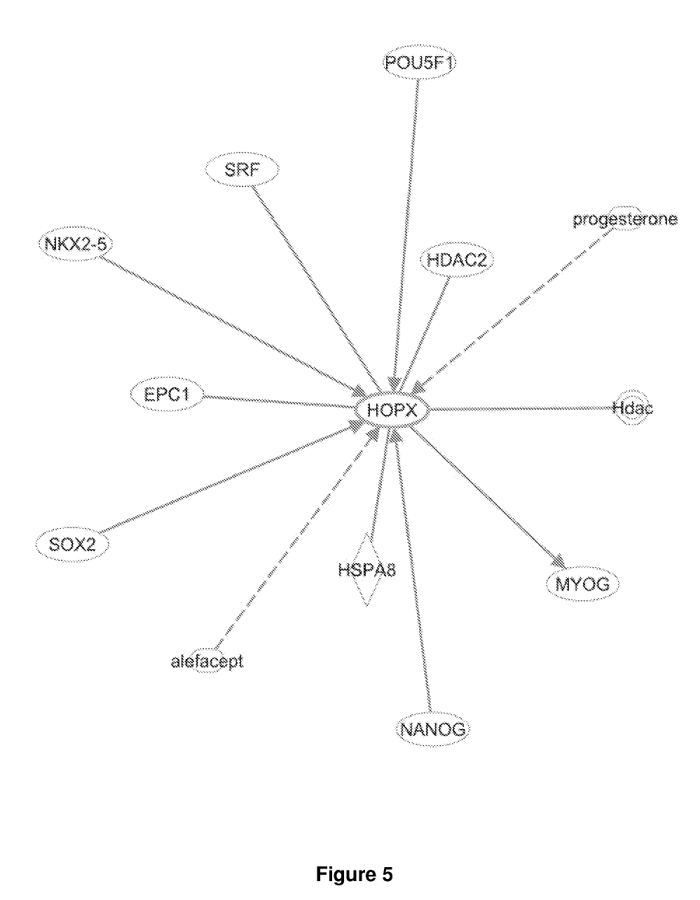

These genes were significantly associated with each other in a network-based analysis (FIG. 4). Interestingly, the homeobox gene HOPX has known interactions with the induced pluripotency factors SOX2, OCT4, and NANOG, as well as the histone deacetylase HDAC2 (FIG. 5). In addition to the anticipated CD34 and CD38 cell surface markers, this group of genes captured several genes known to be differentially expressed in early hematopoiesis including VNN1, RBPMS, SETBP1, GUCY1A3, and MEF2C. Consistently, when evaluated by GSEA, genes differentially expressed between LSC and LPC revealed a highly significant relationship to defined normal hematopoietic precursors (FIG. 1B and Table 2). Genes up-regulated in LSC were highly enriched for those expressed in normal CD34+CD38− cells, containing hematopoietic stem cells (HSC), compared to normal CD34+CD38+ progenitors; and for those preferentially expressed in normal CD133+ cells, also enriched in HSC, compared to CD133− hematopoietic cells. Notably, up-regulated genes were enriched for those associated with AML exhibiting high expression of BAALC, a poor prognostic factor in AML (Mrozek K, et al. Clinical relevance of mutations and gene-expression changes in adult acute myeloid leukemia with normal cytogenetics: are we ready for a prognostically prioritized molecular classification? Blood. Jan. 15, 2007; 109(2):431-448). Conversely, proliferation, cell cycle, and differentiation genes were systematically repressed in the LSC-containing fraction when compared to more mature LPC, consistent with a tendency for replicative quiescence (Dick J E. Stem cell concepts renew cancer research. Blood. Dec. 15, 2008; 112(13):4793-4807).

TABLE 2
Gene sets associated with genes enriched in LSC or LPC. Details of selected gene
sets significantly associated with expression differences between LSC and LPC. The
description displayed in FIG. 1B is given, along with the original source of the gene
set. Original gene set database accession is listed with URL, together with the PubMed
ID for the original publication from which the gene set was derived. Sources of gene
signatures: “mSigDB”: Broad Institute (Subramanian A, et al. Bioinformatics. 2007;
23(23): 3251-3253); “DFCI GenesigDB”: Dana Farber Cancer Institute (Culhane
A C, et al. Nucleic Acids Res. 38(Database issue): D716-725); “SignatureDB”:
Staudt Lab (Shaffer a l, et al. Immunol Rev. 2006; 210: 67-85); or primary literature.
Pubmed ID citations: (7) Georgantas R W, 3rd, et al. Cancer Res. 2004; 64(13):
4434-4441; (8) Toren A, et al. Stem Cells. 2005; 23(8): 1142-1153; (9) Langer C, et
al. Blood. 2008; 111(11): 5371-5379; (10) Su Al, et al. Proc Natl Acad Sci USA.
2004; 101(16): 6062-6067; (11) Venezia T A, et al. PLoS Biol. 2004; 2(10): e301;
(12) Liu D, et al. Proc Natl Acad Sci USA. 2004; 101(19): 7240-7245.

| Description in FIG. 1B | Source | Source accession and link | Pubmed ID |
|---|---|---|---|
| Up in CD34+CD38− | mSigDB | HEMATOP_STEM_ALL_UP | 15231652 |
| vs CD34+CD38+Lin+ | | | |
| Up in CD133+ vs | DFCI GenesigDB | TOREN05_132GENES | 16140871 |
| CD133− | | | |
| Up in high BAALC/ | DFCI GenesigDB | LANGER08_29GENES | 18378853 |
| poor outcome AML | | | |
| Proliferation genes | SignatureDB | PROLIFERATION_NODE1618 | 15075390 |
| HSC proliferation | Literature | Table 10 of PubMed ID reference | 15459755 |
| signature | | | |
| Cell cycle genes | SignatureDB | CELL_CYCLE_LIU | 15123814 |

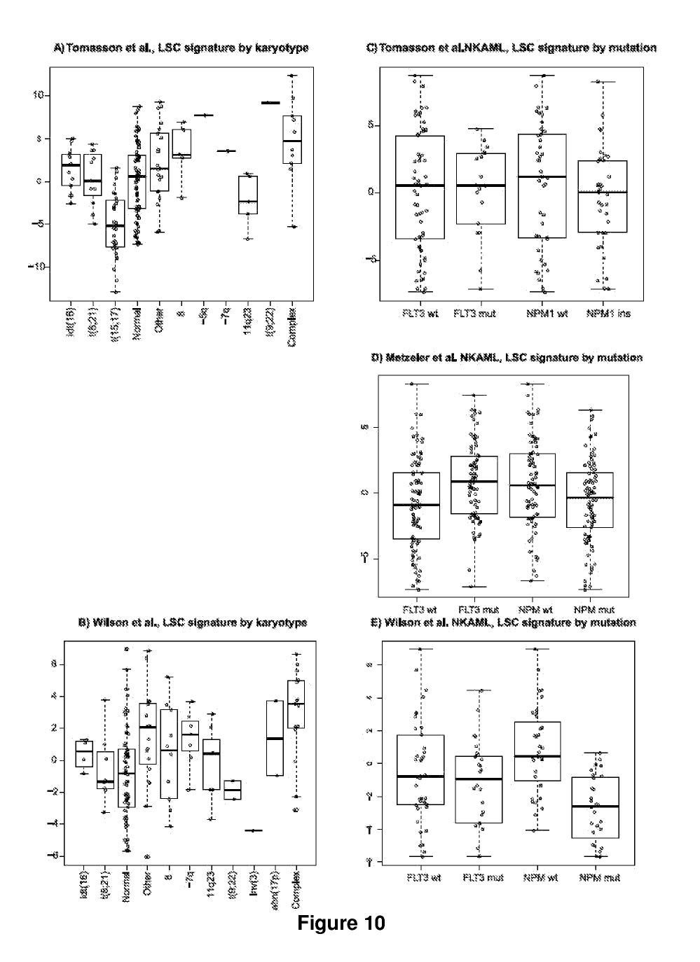

To develop a single metric of LSC gene expression, genes up-regulated in LSC were combined to generate an LSC signature. This signature was assessed in purified cell subsets from primary AML patient samples and across normal human myeloid differentiation. The signature was highly expressed in LSC-enriched populations compared to LPC, but also relative to their progeny CD34− blasts (FIG. 10). Among normal hematopoietic populations from healthy individuals, the LSC signature was high in HSC and multipotent progenitors (MPP), compared to more mature myeloid progenitor populations (FIG. 10). In an independent dataset, the LSC signature was highest in normal CD34+ hematopoietic progenitors relative to normal megakaryoblasts, erythroblasts, myeloblasts, monoblasts, and their mature differentiated progeny, including eosinophils, neutrophils, and monocytes (not shown). These observations, along with GSEA results, indicate that the LSC signature is shared with normal HSC, implying that it may reflect self-renewal ability and relative proliferative quiescence.

An LSC Score Predicts Inferior Survival.

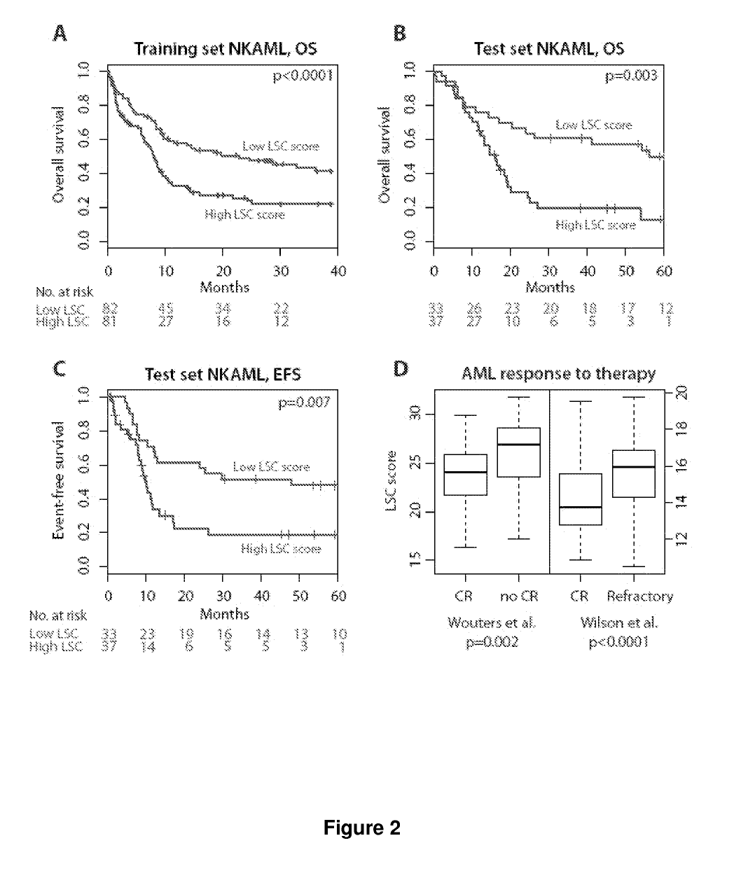

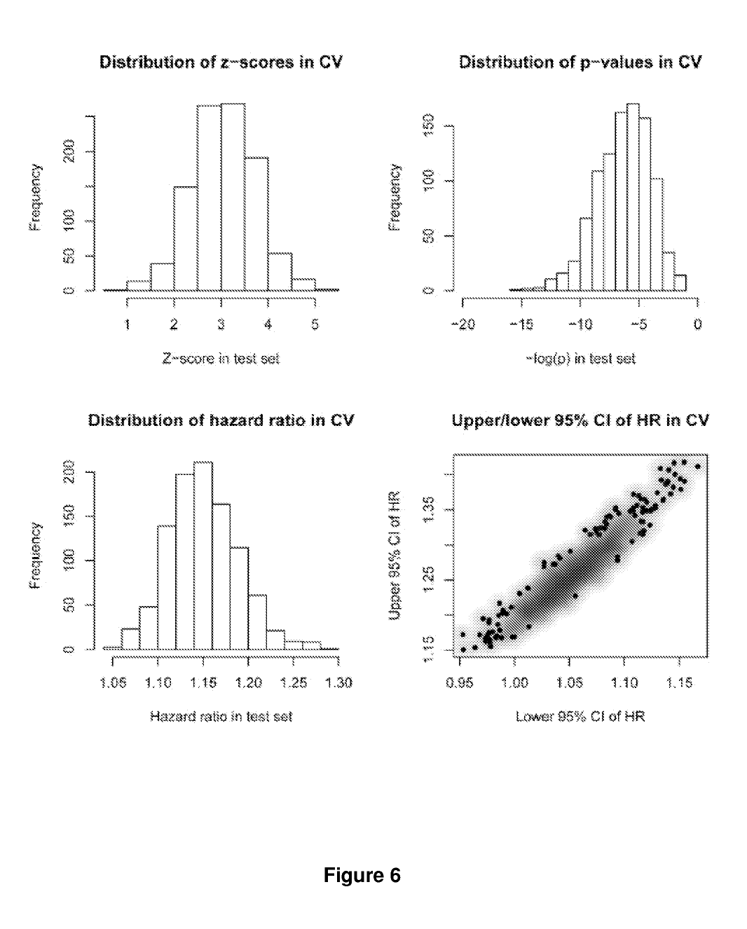

We next evaluated whether expression of LSC signature genes was associated with clinical outcomes using four public datasets of bulk AML expression profiles (Table 9 in Methods section). Since acute promyelocytic leukemia (APL) is a distinct disease entity, it was excluded from all analyses reported. In a training set of n=163 normal karyotype (NKAML patients) (Metzeler K H, et al. An 86-probe-set gene-expression signature predicts survival in cytogenetically normal acute myeloid leukemia. Blood. Nov. 15, 2008; 112(10):4193-4201), an LSC score, defined as a weighted sum (Table 9 in Method section below) of signature genes more highly expressed in the LSC-enriched fraction, was strongly associated with overall survival (OS) [p<0.0001], with higher score predicting inferior outcome (Table 3). The hazard ratio for OS was 1.15 (95% CI 1.08-1.22), with the LSC score ranging from 17.4 to 33.1 (median 24.9). Stratification into groups with higher- or lower- than median LSC scores robustly separated survival curves [p=0.002, HR=1.85 (95% CI 1.25-2.74); Table 3 and FIG. 2A]. Association of these genes with OS was robustly supported by internal cross-validation in the training cohort (FIG. 6).

TABLE 3
The LSC Score as a Univariate Predictor of Survival in AML. Prognostic power of the LSC score, FLT3-ITD mutation
status, NPM1 mutation status, age, and cytogenetic risk are shown for OS, EFS, and RFS for the datasets described.
Shown are the hazard ratios with 95% confidence intervals, and p-value (log-likelihood test).

| | Cohort | | | | | | | |
|---|---|---|---|---|---|---|---|---|
| | | | | | Event-Free | | Relapse-Free | |
| | Overall Survival, NK-AML | | Overall Survival AML* | | Survival NK-AML | | Survival NK-AML | |
| Variable | HR (95% CI) | p | HR (95% CI) | p | HR (95% CI) | p | HR | p |
| Wouters, et al. (test) | n = 99 | | n = 219 | | n = 99 | | n = 85 | |
| LSC score (continuous) | 1.17 (1.07-1.28) | 0.0007 | 1.07 (1.02-1.13) | 0.009 | 1.15 (1.09-1.26) | 0.001 | 1.13 (1.01-1.27) | 0.03 |
| LSC score (dichotomous) | 1.86 (1.15-3.02) | 0.01 | 1.36 (0.98-1.88) | 0.07 | 1.69 (1.07-2.69) | 0.02 | 1.78 (0.99-3.25) | 0.055 |
| FLT3-ITD | 1.84 (1.14-2.98) | 0.012 | 1.82 (1.28-2.59) | <0.001 | 2.12 (1.33-3.38) | 0.001 | 2.80 (1.53-5.14) | 0.0005 |
| NPM1c | 0.76 (0.47-1.23) | 0.26 | 0.86 (0.60-1.22) | 0.39 | 0.92 (0.58-1.47) | 0.74 | 1.09 (0.59-2.02) | 0.79 |
| Age | 1.01 (0.98-1.03) | 0.6 | 1.01 (1.00-1.03) | 0.082 | 1.00 (0.98-1.02) | 0.96 | 0.99 (0.97-1.02) | 0.63 |
| Cytogenetic Risk Group | — | — | 2.04 (1.54-2.69) | <0.001 | — | — | — | — |
| Tomasson, et al. (test) | n = 70 | | n = 137 | | n = 70 | | | |
| LSC score (continuous) | 1.13 (1.04-1.22) | 0.003 | 1.10 (1.04-1.17) | 0.001 | 1.11 (1.03-1.21) | 0.007 | | |
| LSC score (dichotomous) | 2.70 (1.43-5.10) | 0.002 | 2.01 (1.27-3.18) | 0.003 | 2.39 (1.26-4.52) | 0.006 | | |
| FLT3-ITD | 2.68 (1.42-5.07) | 0.002 | 1.82 (1.09-3.02) | 0.019 | 2.44 (1.23-4.82) | 0.008 | | |
| NPM1c | 1.55 (0.86-2.79) | 0.14 | 1.49 (0.95-2.32) | 0.079 | 1.29 (0.70-2.37) | 0.41 | | |
| Age | 1.02 (1.00-1.04) | 0.06 | 1.03 (1.01-1.04) | <0.001 | 1.01 (0.99-1.03) | 0.25 | | |
| Cytogenetic Risk Group | — | — | 1.97 (1.34-2.90) | <0.001 | — | — | | |
| Wilson, et al. (test) | n = 65 | | n = 170 | | | | | |
| LSC score (continuous) | 1.18 (1.04-1.34) | 0.011 | 1.15 (1.07-1.25) | <0.001 | | | | |
| LSC score (dichotomous) | 2.55 (1.44-4.51) | <0.001 | 1.99 (1.43-2.79) | 4.00E−05 | | | | |
| FLT3-ITD | 1.28 (0.73-2.23) | 0.39 | 1.12 (0.78-1.62) | 0.53 | | | | |
| NPM1c | 0.67 (0.38-1.17) | 0.16 | 0.79 (0.55-1.14) | 0.2 | | | | |
| Age | 1.03 (1.00-1.05) | 0.029 | 1.03 (1.02-1.05) | <0.001 | | | | |
| Cytogenetic Risk Group | — | — | 2.16 (1.52-3.06) | <0.001 | | | | |
| Metzeler, et al. (training) | n = 163 | | | | | | | |
| LSC score (continuous) | 1.15 (1.08-1.22) | <0.001 | | | | | | |
| LSC score (dichotomous) | 1.85 (1.25-2.74) | 0.002 | | | | | | |
| FLT3-ITD | 2.22 (1.49-3.31) | <0.001 | | | | | | |
| NPM1c | 0.79 (0.54-1.17) | 0.24 | | | | | | |
| Age | 1.03 (1.01-1.04) | <0.001 | | | | | | |
| Cytogenetic Risk Group | — | — | | | | | | |

*Patients with APL were excluded.

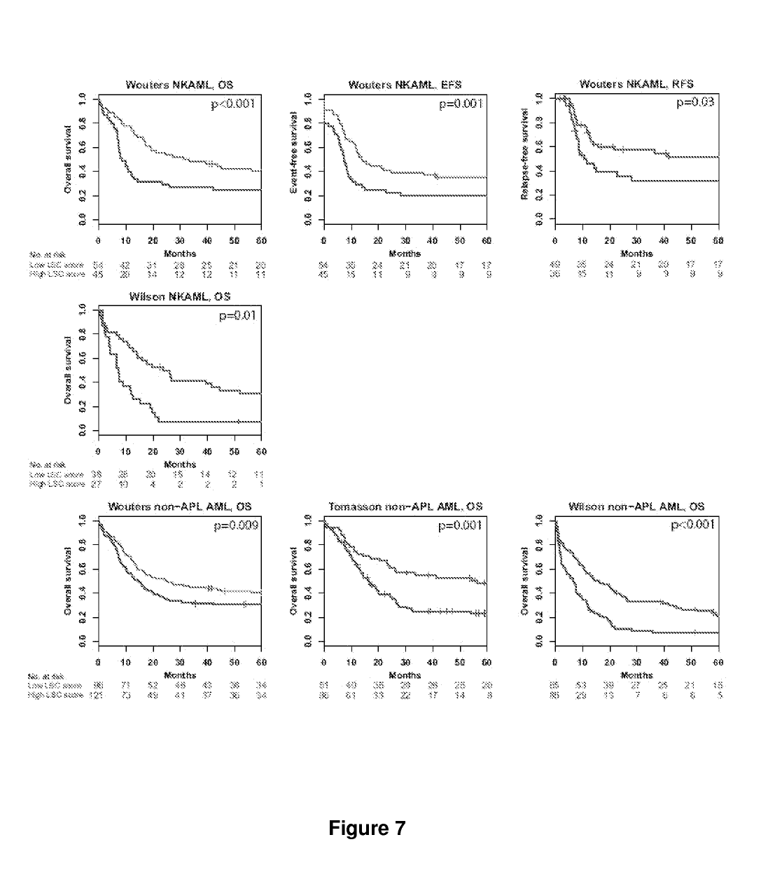

When the same gene weightings were applied to NKAML from three independent datasets, high LSC score was associated with inferior OS as a continuous variable [p<0.012 in all cases, HR from 1.13 to 1.18; Table 3]. Using the median level within the training set as a threshold, stratification into high- or low-LSC groups significantly separated survival curves in each cohort (Table 3, FIGS. 2B and 7). In NKAML patients from one well-characterized cohort of adult AML patients with diverse karyotypes primarily treated with induction regimens including cytarabine and an anthracycline17, 24, the LSC score ranged from 16.6 to 31.0 (see Table 4 below for all ranges) and was predictive of OS (Table 3 and FIG. 2B). This association was significant whether the LSC score was evaluated as a continuous predictor (HR 1.13; p=0.003), or a dichotomous one (HR 2.7; p=0.002), with those in the low group having a median OS of 56.3 months compared to 16.5 months for those in the high group.

TABLE 4
Range of LSC scores across bulk AML datasets used in survival analyses. The mean,
minimum, and maximum LSC score is reported for each of the four bulk AML cohorts,
separately for NKAML and non-APL subsets. As mentioned in the methods section
below, the dataset of Wilson et al. does not have probes for some of the LSC
genes; hence the range is different from the other three datasets.

| | | | Median | | Comparative Absolute | |
|---|---|---|---|---|---|---|
| | | | Survival by | | Risk of Event by | |
| | | | LSC Score | | LSC Score Group | |
| | No. of | LSC Score, | Group, mo | | at 3 y, % (95% CI) | |
| AML Cohort^{b} | Patients | Median (IQR) | Low | High | Low | High |

Normal Karyotype AML

| | | | | | | |
|---|---|---|---|---|---|---|
| Metzeler et al^{16, 27} | 163 | 24.9 (22.6-27.0) | 22.8 | 7.9 | 57 (43-67) | 78 (66-86) |
| Tomasson et al^{17, 20} | 74 | 25.2 (22.3-27.6) | | | | |
| Overall survival | 70 | | 56.3 | 16.3 | 39 (20-54) | 81 (61-90) |
| Event-free survival | 70 | | 47.7 | 9.9 | 48 (27-63) | 81 (60-91) |
| Wouters et al^{19, 26} | 181 | 25.0 (22.6-27.0) | | | | |
| Overall survival | 99 | | 31.3 | 8.4 | 52 (36-63) | 73 (57-84) |
| Event-free survival | 99 | | 14.0 | 7.4 | 61 (46-72) | 80 (64-89) |
| Relapse-free survival | 85 | | 65.6 | 10.4 | 43 (26-56) | 68 (46-81) |
| Wilson et al^{18, C} | 65 | 14.0 (12.8-15.6) | 23.7 | 7.3 | 58 (39-72) | 93 (72-98) |

Non-APL AML

| | | | | | | |
|---|---|---|---|---|---|---|
| Tomasson et al^{17, 20} | 143 | 25.7 (23.1-28.4) | 56.3 | 16.5 | 45 (30-57) | 75 (64-83) |
| Wouters et al^{19, 26} | 392 | 25.6 (23.4-28.2) | 25.0 | 14.5 | 55 (44-64) | 69 (59-76) |
| Wilson et al^{18, C} | 170 | 14.7 (13.4-16.3) | 15.9 | 6.6 | 67 (56-76) | 93 (84-97) |

Including all non-APL patients, the LSC score varied from 16.6 to 35.5 in this cohort, and each incremental unit increased the HR for death by 1.10 fold (p=0.001). Patients in the low LSC signature group had a median OS of 56.3 months compared to 16.5 months in the high group (HR 2.0 (95% CI 1.3-3.2); p=0.003). Investigation of the LSC score in non-APL patients from two additional cohorts including patients with cytogenetic abnormalities confirmed its association with adverse OS in both (FIG. 7 and Table 3).

Higher LSC Score Predicts Inferior EFS, Refractoriness to Treatment, and Disease Relapse.

Higher LSC scores were consistently associated with inferior EFS in NKAML patients (p<0.008 in all cases, HR from 1.11 to 1.15 for continuous LSC score; Table 3). As with OS, the LSC-high group had inferior EFS (FIG. 2C), with a median of 10 months compared to 48 months in the LSC-low group. The LSC score was predictive of EFS in the Wouters et al. dataset (Wouters et al. Double CEBPA mutations, but not single CEBPA mutations, define a subgroup of acute myeloid leukemia with a distinctive gene expression profile that is uniquely associated with a favorable outcome. Blood. Mar. 26, 2009; 113(13):3088-3091) (Table 3; HR 1.15; p=0.001), and high/low LSC grouping separated survival curves (FIG. 7; HR=1.7, p=0.02). For the Wouters et al. dataset, LSC scores also predicted RFS in NKAML (p=0.03, HR=1.1; Table 3 and FIG. 7), with median RFS of 66 months in the low-LSC score cases, and 10 months in the high-LSC group.

Early divergence of the survival curves of the LSC-high and low groups suggested association of the LSC score with initial therapeutic response. Consistent with this, the rate of clinical remission (CR) was superior among AML patients with low LSC score compared to those in the high group, both in an older cohort (Wilson et al. cohort (Wilson C S, et al. Gene expression profiling of adult acute myeloid leukemia identifies novel biologic clusters for risk classification and outcome prediction. Blood. Jul. 15, 2006; 108(2):685-696): median age 65 y, 56% CR vs. 29%, p<0.001 by Fisher exact test), and a younger one (Wouters et al. cohort (Wouters B J, et al. Double CEBPA mutations, but not single CEBPA mutations, define a subgroup of acute myeloid leukemia with a distinctive gene expression profile that is uniquely associated with a favorable outcome. Blood. Mar. 26, 2009; 113(13):3088-3091; Valk P J, et al. Prognostically useful gene-expression profiles in acute myeloid leukemia. N Engl J Med. Apr. 15, 2004; 350(16):1617-1628): median age 43 y, 88% vs. 76%, p=0.02). Correspondingly, LSC scores were significantly higher in patients failing to achieve CR compared to those who did (p=0.002 and p<0.0001 for the two cohorts), a distinction most evident for those patients in whom such remissions were durable (FIG. 2D; p=0.002).

Lower Expression of the LSC Score Among Prognostically Favorable Groups.

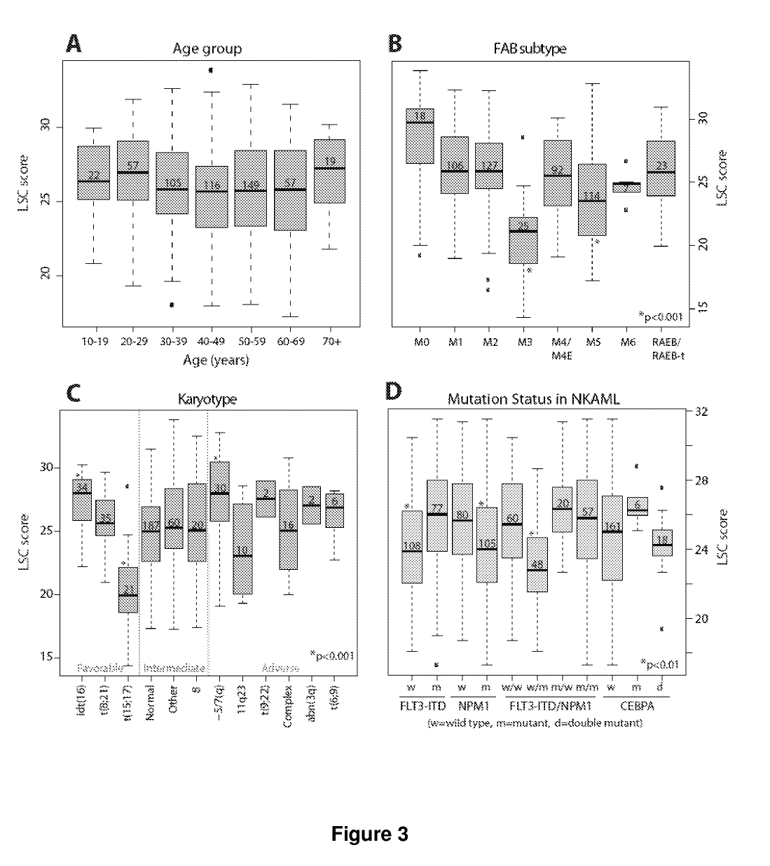

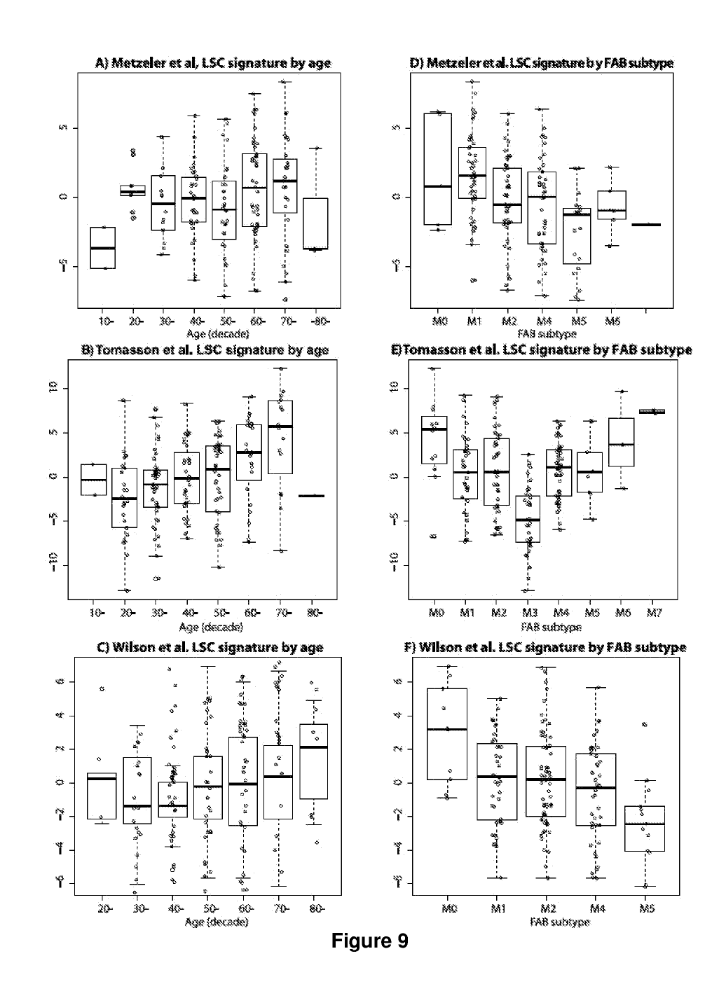

Extensive clinical investigation in adult AML has defined several important prognostic factors including age, karyotype and molecular mutations, particularly internal tandem duplications in FLT3 (FLT3-ITD) and mis-localizing mutations in NPM1 (NPM1c) (Grimwade D, Hills R K. Independent prognostic factors for AML outcome. Hematology Am Soc Hematol Educ Program. 2009:385-395; Mrozek K, et al. Clinical relevance of mutations and gene-expression changes in adult acute myeloid leukemia with normal cytogenetics: are we ready for a prognostically prioritized molecular classification? Blood. Jan. 15, 2007; 109(2):431-448). The association of the LSC score with these risk factors was assessed in the Metzler et al, Tomasson et al., Wilson et al. and Wouters et al. AML cohorts. Though similar across most age groups (FIGS. 3A, 9), and among morphological subtypes (FIGS. 3B and 9), LSC scores were significantly lower in the M3 group (APL) than other subtypes (FIGS. 3B and 9). Notably, cases with minimally differentiated myeloblasts (MO), typically lacking expression of myeloperoxidase and having poor prognosis (Lee E J, et al. Minimally differentiated acute nonlymphocytic leukemia: a distinct entity. Blood. November 1987; 70(5):1400-1406), showed particularly high LSC scores (FIGS. 3B and 9), consistent with previous reports of high LSC prevalence in this subtype (Payton J E, et al. High throughput digital quantification of mRNA abundance in primary human acute myeloid leukemia samples. J Clin Invest. June 2009; 119(6):1714-1726). In agreement with our observation of low LSC scores in APL, among AML with recurrent karyotypic anomalies, those harboring the t(15;17)(q22;q21) translocation had the lowest scores (FIGS. 3C and 10). Notably, APL is distinct among most FAB subgroups of AML, as the identity of LSC has yet to be definitively characterized (Bonnet D, Dick J E. Human acute myeloid leukemia is organized as a hierarchy that originates from a primitive hematopoietic cell. Nat. Med. July 1997; 3(7):730-737; Ishikawa F, et al. Chemotherapy-resistant human AML stem cells home to and engraft within the bone-marrow endosteal region. Nat Biotechnol. November 2007; 25(11):1315-1321). Across most other cytogenetic subgroups, the LSC score was similar with the exception of higher than average values in patients with unfavorable −5 or 7(q) abnormalities, and lower than average values among AML harboring anomalies involving 11q23/MLL (FIGS. 3C and 10). The latter is consistent with recent studies, reporting that self-renewing cells from AML mouse models carrying MLL anomalies reside in more mature cells (Cleary M L. Regulating the leukaemia stem cell. Best Practice & Research Clinical Haematology. 2009; 22(4):483-487).

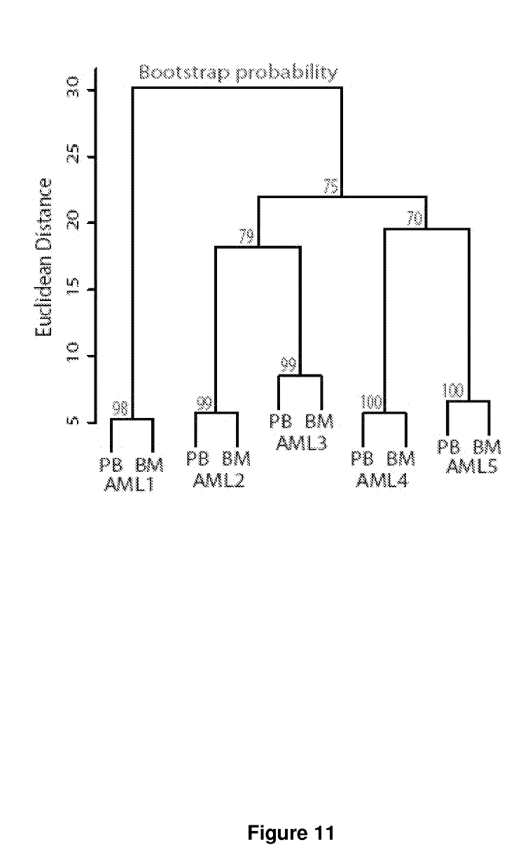

We also investigated the relationship of the LSC score to molecular mutations in the largest single cytogenetic subgroup of AML, NKAML. LSC scores were significantly lower in those harboring NPM1c mutations (FIGS. 3D and 10)), consistent with recent observations that leukemia initiating cells in NPM1 mutant AML are frequently CD34 negative (Taussig D C, et al. Leukemia initiating cells from some acute myeloid leukemia patients with mutated nucleophosmin reside in the CD34− fraction. Blood. Jan. 6, 2010). Furthermore, LSC scores were significantly lower within the subgroup of patients with wild type FLT3 but mutant NPM1c, a combination conferring a distinctly favorable prognosis in NKAML (FIG. 3D) (Schlenk R F, et al. Mutations and treatment outcome in cytogenetically normal acute myeloid leukemia. N Engl J Med. May 1, 2008; 358(18):1909-1918). LSC scores were also lower in NKAML with double CEBPA mutations, also associated with favorable outcomes (FIG. 3D) (Wouters B J, 2009, supra)., relative to cases with single mutants, but not relative to wild-type CEBPA. Similar findings were observed in all four independent datasets totaling 1047 patients (FIG. 10). Of note, no significant differences in LSC scores were observed when patients with AML were stratified according to less common recurrent somatic mutations, including those in the tyrosine kinase domain of FLT3 (FLT3-TKD), or activating mutations in NRAS, KRAS, or IDH1. Finally, expression of LSC score genes was not dependent on tissue-of-origin (peripheral blood vs. bone marrow) in bulk AML samples (FIG. 11).

The LSC Score is Independently Prognostic.

In order to test whether the LSC score added to established clinical predictors of risk such as age, cytogenetics, and molecular mutations (FLT3-ITD and NPM1), we used multivariate Cox regression. The LSC score made a significant independent contribution to predicting OS and EFS in NKAML and across non-APL AML in all but one instance (Table 5). Comparison of AUC (Area Under the Curve) for Receiver Operator Characteristic (ROC) curves showed that the LSC score added to the prognostic utility of age, FLT3-ITD, NPM1c, and cytogenetic risk in predicting OS at 2 years in all cohorts for both NKAML and non-APL AML (Table 6). In NKAML, higher LSC score associated with inferior OS in NPM1-mutant cases, despite the fact that they are frequently CD34-negative, and in patients with both wild-type FLT3 and wild-type NPM1 (Table 7). Furthermore, when all analyses (including derivation of LSC score weightings) were performed excluding CD34-negative cases (NPM1 mutant; or the 40% of samples with lowest CD34 expression), similar findings were noted. Exclusion of CD34 from the model-building and validation resulted in an LSC score with similar gene weightings and prognostic capability. Taken together, these data indicate that higher LSC score is predictive of inferior survival outcomes independent of age, FLT3-ITD, NPM1 mutations, CD34 expression, and cytogenetic risk group, and adds to their prognostic utility.

TABLE 5
Multivariate Survival Prediction Including the LSC Score in AML. The LSC score was tested as a multivariate
predictor in combination with FLT3-ITD status, NPM1 status, age, and cytogenetic risk group using Cox regression.
Hazard ratios and p-values (log-likelihood test) are reported for each variable within the multivariate model.
The overall log-likelihood p-value for the model is also indicated. The number of patients (n) differs from
those in Table 3 depending on whether information on all covariates was available in each case.

| | Cohort | | | | | | | |
|---|---|---|---|---|---|---|---|---|
| | Overall Survival, NK-AML | | Overall Survival AML* | | EFS NK-AML | | RFS NK-AML | |
| Variable | HR (95% CI) | p | HR (95% CI) | p | HR (95% CI) | p | HR (95% CI) | p |
| Wouters, et al. | n = 99 | | n = 219 | | n = 99 | | n = 85 | |
| LSC score | 1.16 (1.05-1.27) | 0.003 | 1.07 (1.01-1.13) | 0.02 | 1.14 (1.04-1.24) | 0.004 | 1.12 (1.00-1.25) | 0.05 |
| FLT3-ITD | 1.94 (1.15-3.27) | 0.013 | 1.98 (1.35-2.91) | <0.001 | 2.15 (1.30-3.55) | 0.003 | 2.83 (1.47-5.45) | 0.002 |
| NPM1c | 0.73 (0.42-1.27) | 0.27 | 0.70 (0.46-1.06) | 0.094 | 0.88 (0.52-1.48) | 0.62 | 0.95 (0.47-1.91) | 0.89 |
| Age | 1.02 (1.00-1.04) | 0.087 | 1.02 (1.00-1.03) | 0.023 | 1.01 (0.99-1.03) | 0.35 | 1.00 (0.98-1.03) | 0.78 |
| Cytogenetic Risk Group | — | — | 2.02 (1.53-2.67) | <0.001 | — | — | — | — |
| Overall | | <0.001 | | <0.001 | | <0.001 | | 0.004 |
| Tomasson, et al. | n = 70 | | n = 137 | | n = 70 | | | |
| LSC score | 1.15 (1.06-1.26) | 0.002 | 1.10 (1.03-1.17) | 0.005 | 1.13 (1.04-1.23) | 0.005 | | |
| FLT3-ITD | 3.00 (1.50-6.00) | 0.002 | 2.00 (1.18-3.37) | 0.01 | 2.86 (1.37-5.94) | 0.005 | | |
| NPM1c | 1.58 (0.83-3.01) | 0.17 | 1.64 (1.01-2.65) | 0.045 | 1.27 (0.67-2.42) | 0.46 | | |
| Age | 1.02 (1.00-1.04) | 0.14 | 1.02 (1.01-1.04) | 0.007 | 1.01 (0.99-1.03) | 0.38 | | |
| Cytogenetic Risk Group | — | — | 1.86 (1.26-2.76) | 0.002 | — | — | | |
| Overall | | <0.001 | | <0.001 | | 0.003 | | |
| Wilson, et al. | n = 63 | | n = 136 | | | | | |
| LSC score | 1.14 (0.97-1.34) | 0.1 | 1.17 (1.05-1.30) | 0.005 | | | | |
| FLT3-ITD | 2.05 (1.10-3.85) | 0.025 | 1.45 (0.91-2.30) | 0.12 | | | | |
| NPM1c | 0.82 (0.42-1.61) | 0.57 | 0.93 (0.55-1.60) | 0.8 | | | | |
| Age | 1.03 (1.00-1.06) | 0.026 | 1.03 (1.01-1.04) | 0.002 | | | | |
| Cytogenetic Risk Group | — | — | 1.99 (1.37-2.89) | <0.001 | | | | |
| Overall | | 0.01 | | <0.001 | | | | |
| Metzeler, et al. | n = 162 | | | | | | | |
| LSC score | 1.10 (1.03-1.17) | 0.006 | | | | | | |
| FLT3-ITD | 2.19 (1.42-3.37) | <0.001 | | | | | | |
| NPM1c | 0.87 (0.58-1.30) | 0.49 | | | | | | |
| Age | 1.03 (1.01-1.04) | <0.001 | | | | | | |
| Cytogenetic Risk Group | — | — | | | | | | |
| Overall | | <0.001 | | | | | | |

*Patients with APL were excluded.

TABLE 6
Area under curve (AUC) of receiver-operating characteristic curves
for model predictions of overall survival at 2 years. AUC was
calculated for ROC curves for models predicting OS at 2 years,
as defined in the training set (Metzeler for NKAML, Tomasson for
non-APL) and applied to the test sets. Reported are the AUC values
for the LSC score alone in NKAML and non-APL, LSC score combined
with Age, FLT3-ITD and NPM1 status in NKAML, and all of these
variables together with cytogenetic risk group in non- APL. Higher
AUC values indicate better model performance.
NKAML

| | LSC | Age + FLT3 + | Age + FLT3 + |
|---|---|---|---|
| | score | NPM1 | NPM1 + LSC group |
| Metzeler (train) | 0.70 | 0.77 | 0.78 |
| Tomasson (test) | 0.73 | 0.70 | 0.76 |
| Wouters (test) | 0.67 | 0.64 | 0.68 |
| Wilson (test) | 0.74 | 0.74 | 0.82 |

non-APL

| | | | Age + CytoRisk + |
|---|---|---|---|
| | LSC | Age + CytoRisk + | FLT3 + NPM1 + |
| | score | FLT3 + NPM1 | LSC group |
| Tomasson (train) | 0.62 | 0.75 | 0.77 |
| Wouters (test) | 0.60 | 0.65 | 0.69 |
| Wilson (test) | 0.69 | 0.77 | 0.84 |

TABLE 7
Performance of LSC score as a continuous variable predicting overall
survival in subsets of NKAML. Performance of the LSC score in
the CD34-negative NPM1-mutant (irrespective of FLT3 status) subsets
of NKAML is shown, together with performance within NKAML subsets
harboring wild-type FTL3-ITD and NPM1. The latter are the most
homogeneous sets of patients for which sufficient numbers of samples
were available to analyze survival outcomes.

| | NPM1 mutant | | NPM1wt/FLT3wt | |
|---|---|---|---|---|
| | Cohort | | | |
| | Overall Survival, NK-AML | | Overall Survival, NK-AML | |
| Variable | HR (95% CI) | p | HR (95% CI) | p |
| | n = 61 | | n = 27 | |
| Wouters, et al. | 1.15 (1.03-1.30) | 0.017 | 1.20 (0.98-1.48) | 0.073 |
| | n = 33 | | n = 32 | |
| Tomasson, | 1.12 (0.99-1.27) | 0.07 | 1.16 (1.02-1.32) | 0.016 |
| et al. | | | | |
| | n = 28 | | n = 24 | |
| Wilson, et al. | 1.23 (0.90-1.69) | 0.18 | 1.08 (0.86-1.35) | 0.5 |
| | n = 86 | | n = 47 | |
| Metzeler, et | 1.10 (1.01-1.20) | 0.037 | 1.20 (1.07-1.36) | 0.002 |
| al. (training) | | | | |

### Discussion

Clinical evidence supporting the significance of the cancer stem cell model for human AML has been lacking despite ample experimental evidence in its support from transplantation assays in immunocompromised mice. Here we show that a gene expression score associated with the LSC-enriched subpopulation is an independent prognostic factor in AML, with high score predictive of adverse outcomes in multiple independent cohorts. Specifically, high LSC score is predictive of poor OS, EFS, and RFS in NKAML, and inferior OS in patients with karyotypic anomalies. Additionally, the LSC score was associated with primary response to induction chemotherapy, as high scores strongly correlated with refractoriness to remission. Multivariate analysis demonstrated that this signature predicted poor outcomes independently of age, FLT3 or NPM1 mutations, and cytogenetic risk group. These findings support the clinical relevance of the cancer stem cell model for AML.

The majority of reports indicate that LSC activity is enriched in the CD34+CD38− fraction (Dick J E. Stem cell concepts renew cancer research. Blood. Dec. 15, 2008; 112(13):4793-4807), although recent studies have identified such activity in additional populations (Taussig D C, et al. Anti-CD38 antibody-mediated clearance of human repopulating cells masks the heterogeneity of leukemia-initiating cells. Blood. Aug. 1, 2008; 112(3):568-575; Taussig D C, et al. Leukemia initiating cells from some acute myeloid leukemia patients with mutated nucleophosmin reside in the CD34− fraction. Blood. Jan. 6, 2010). In the current study, LSC were defined as CD34+CD38−, while LPC were defined as CD34+CD38+. In half of the studied cases, these definitions were directly confirmed by transplantation assays, and while the other samples failed to engraft, paired samples from all profiled patients exhibited coherence of the LSC expression profile across all patients. Notably, the LSC signature was highly expressed in purified HSC, and much lower in myeloid progenitors, suggesting that it may be reflective of self-renewal ability.

The higher expression of the LSC signature within HSC may reflect more limited self-renewal potential of LSC as compared with HSC, or relate to heterogeneity of the CD34+CD38− leukemic population, with bona fide AML-initiating cells comprising a subset of this population. The observed similarities between the LSC signature and HSC gene expression programs do not preclude therapeutic targeting of leukemic stem cells without untoward toxicity affecting normal hematopoiesis. Indeed, markers distinguishing LSC from HSC exist and are amenable to targeted therapies (Nitta T, Takahama Y. The lymphocyte guard-IANs: regulation of lymphocyte survival by IAN/GIMAP family proteins. Trends in Immunology. 2007; 28(2):58-65; Guenther M G, et al. A Chromatin Landmark and Transcription Initiation at Most Promoters in Human Cells. Cell. 2007; 130(1):77-88; Kook H, et al. Cardiac hypertrophy and histone deacetylase-dependent transcriptional repression mediated by the atypical homeodomain protein Hop. The Journal of Clinical Investigation. 2003; 112(6):863-871).

In addition to the markers employed for their purification (CD34 and CD38), and others known to be differentially expressed during early myelopoiesis, LSC were distinguished from LPC in their expression of several genes. These included three members (GIMAP2, GIMAP6, and GIMAP7) of a small family of immune-associated nucleotide-binding proteins implicated in survival of hematopoietic cells and leukemia (Nitta T, Takahama Y. The lymphocyte guard-IANs: regulation of lymphocyte survival by IAN/GIMAP family proteins. Trends in Immunology. 2007; 28(2):58-65); however, no prior associations with AML have been described. Two genes, HOPX, GUCY1A3, (FIG. 4) in this signature are notable for their distinctive pattern of expression and histone modification in self-renewing cells (Guenther M G, et al. 2007 supra). HOPX is an unusual homeodomain protein known to directly recruit histone deacetylase activity without directly binding DNA (Kook H, et al. 2003, supra) and to be directly repressed in vivo in malignant cells in response to administration of the histone deacetylase inhibitor panobinostat (Ellis L, et al. Histone Deacetylase Inhibitor Panobinostat Induces Clinical Responses with Associated Alterations in Gene Expression Profiles in Cutaneous T-Cell Lymphoma. Clinical Cancer Research. Jul. 15, 2008 2008; 14(14):4500-4510). The latter is currently being studied in clinical trials for patients with AML. GUCY1A3, which encodes a component of the soluble guanylate cyclase enzyme catalyzing the conversion of GTP to cGMP, is repressed during replicative senescence (Lodygin D, et al. Induction of the Cdk inhibitor p21 by LY83583 inhibits tumor cell proliferation in a p53-independent manner. The Journal of Clinical Investigation. 2002; 110(11):1717-1727), and cGMP has been reported to stimulate HSC proliferation (Oshita A, et al. cGMP stimulation of stem cell proliferation. Blood. 1977; 49(4):585-591).

Our study is the first to directly define a signature of enriched AML-initiating cells, and to relate this signature to expression profiles of diagnostic specimens, allowing a link to corresponding clinical and pathological features of patients. Ultimately, this model has major implications for cancer therapy, most notably that in order to achieve cure, the cancer stem cells must be eliminated. To accomplish this in AML, novel therapies targeting LSC must be developed. Several such therapies are being investigated including small molecules (Guzman, M L et al. The sesquiterpene lactone parthenolide induces apoptosis of human acute myelogenous leukemia stem and progenitor cells. Blood. 2005. 105(11):4163-4169; Guzman, M L et al. Rapid and selective death of leukemia stem and progenitor cells induced by the compound 4-benzyl, 2-methyl, 1,2,4-thiadiazolidine, 3,5 dione (TDZD-8). Blood. 2007. 110(13):4436-4444; Hahn, C K et al. Proteomic and genetic approaches identify Syk as an AML target. Cancer Cell. 2009. 16(4):281-294; Hassane, D C et al. Discovery of agents that eradicate leukemia stem cells using an in silico screen of public gene expression data. Blood. 2008. 111(12):5654-5662) and monoclonal antibodies (Jin, L. et al. Monoclonal antibody-meidated targeting of CD123, IL-3 receptor alpha chain, eliminates human acute myeloid leukemic stem cells. Cell Stem Cell. 2009. 5(1):31-42; Majeti, R. et al. CD47 is an adverse prognostic factor and therapeutic antibody target on human acute myeloid leukemia stem cells. Cell. 2009. 138(2):286-299; Jin, L. et al. Targeting of CD44 eradicates human acute myeloid leukemic stem cells. Nat. Med. 2006. 12(1):1167-1174) which hold promise for improving therapeutic efficacy beyond current conventional therapies.

### Materials and Methods

Cellular Fractionation and Expression Profiling of Normal and Leukemic Subsets.

Human samples were obtained at the Stanford University Medical Center (SUMC) according to an approved protocol of the Institutional Review Board (IRB) after informed consent. Normal human bone marrow (BM) mononuclear cells were purchased from AllCells Inc. (Emeryville, Calif.) and human cord blood (CB) was obtained from SUMC. For AML specimens, peripheral blood and/or bone marrow was obtained, and gene expression microarray data were generated using Affymetrix U133 Plus 2.0 microarrays from the following FACS-purified populations: AML LSC (Lin-CD34+CD38−), AML LPC (Lin-CD34+CD38+), AML Blasts (Lin-CD34−), HSC (Lin-CD34+CD38−CD90+CD45RA−; BM and CB, n=7), MPP (Lin-CD34+CD38−CD90−CD45RA−; BM and CB, n=7), CMP (Lin-CD34+CD38+CD123+CD45RA−; BM, n=4), GMP (Lin-CD34+CD38+CD123+CD45RA+; BM, n=4), and MEP (Lin-CD34+CD38+CD123−CD45RA−; BM, n=4). Detailed methods for purification of cellular subsets and clinical features of the corresponding AML patients have been reported previously (Majeti R, et al. Proc Natl Acad Sci USA 2009; 106:3396-401).

Sample Annotations.

Clinical covariates corresponding to expression arrays were obtained from NCBI GEO and caArray as described below and summarized in Table 8. The largest cohort discussed (n=526, Wouters et al., GEO accession GSE14468) included a subset (n=295) which had been discussed in a separate publication (Valk et al. NEJM 2004) with clinical annotations in the publication supplementary material. We merged the available annotations from these two publications for the overlapping samples.

TABLE 8
Bulk AML public datasets used. Summary of cohort information for the four public AML datasets used. Included
are the corresponding cooperative groups, primary author of publications, journal citation, and PubMed ID.
Cohort summary information indicates size of study, type of AML samples, and age of patients (median and range).
Microarray platform and database accession (GEO or caArray) are indicated, along with available demographic
and hematopathologic information. We also summarize the molecular data collected (mutations), primary therapy
protocol, and survival data available for each study (response to therapy, OS, EFS, RFS).

| | Dataset 1 | Dataset 2 | Dataset 3 | Dataset 4 |
|---|---|---|---|---|
| Primary Author | Wouters, B J, et al.; | Metzeler, K H, et al.; | Tomasson, M H, et al.; | Wilson, C S, et al. |
| | Valk, P J, et al. | Dufour, A, et al. | Mardis, E R, et al. | |
| Citation | (2009) Blood 113: 3088; | (2008) Blood 112: 4193; | (2006) Blood 111: 4797; | (2006) Blood 108: 685 |
| | (2004) NEJM 350: 1617 | (2009) J Clin Oncol | (2008) NEJM 361: 1058 | |
| PubMed ID | 19171880, 15084694 | 18716133 | 18270328 | 16597596 |
| Cohort | Adult AML, Mixed | Adult AML, Normal | Adult AML, Mixed | Adult AML, Mixed |
| | karyotypes | karyotype | karyotypes | karyotypes |
| Median age-yrs, | 46 (15-77) | 60 (17-85) | 47 (16-81) | 65 (20-84) |
| (range) | | | | |
| Patients (n) | 526 | 163 | 188 | 170 |
| Cooperative | Dutch-Belgian | German AML | Washington University | Southwest Oncology |
| Group | Hematology-Oncology | Cooperative Group | (WU) and CALGB | Group (SWOG) |
| | Cooperative (HOVON) | (AMLCG) | | |
| Microarray | Affymetrix HG-U133 Plus | Affymetrix HG-U133 A&B | Affymetrix HG-U133 Plus | Affymetrix HG-U95Av2 |
| Platform(s) | 2.0 | | 2.0 | |
| Dataset | GSE14468 | GSE12417 | GSE10358 | NCI-caArray-willm-00119 |
| Accession | | | | |
| Demographic | Age; Gender | Age: Gender | Age; Gender; Race | Age; Gender; |
| Data | | | | Preceding malignancy |
| Hematopathology | Tissue Source; FAB; | Tissue Source; FAB; | Tissue Source; FAB; | Tissue Source; FAB; |
| | Karyotype | Karyotype | Karyotype; WBC; BM % | Karyotype |
| | | | Blasts | |
| Molecular | FLT3; RAS; EVI1; | FLT3; NPM1 | 26 tyrosine kinase genes; | FLT3; NPM1 |
| Testing | CEBPA | | FLT3; NPM1; CEBPA; | |
| | | | WT1; KIT; NRAS; IDH1; | |
| | | | ND4; NUP98; NSD1; | |
| | | | ETV6; PTPN11; TP53; | |
| | | | RUNX1; SPI1 | |
| Primary Therapy | Multiple HOVON | AMLCG 1999 | WashU: Primarily 7 + 3; | S9031/S9333/S9034/ |
| Protocol(s) | trials: PMIDs 9396403, | | CALGB | S9500/S9126 |
| | 12930926, 15070662 | | 9621/9222/9191/9710 | |
| Patient Outcome | Response to Primary | OS | OS; EFS | Response to Primary |
| Data | Therapy; OS; EFS; RFS | | | Therapy; OS |

Selection of Non-APL and NKAML Subsets.

The Metzeler et al. dataset contains only NKAML data, with no cases of APL (no sample selection was necessary). All 163 samples had available OS time and status. Wilson et al. consists entirely of non-APL, but a mixture of FAB subtypes and karyotypic groups. For this dataset, in selecting NKAML we filtered for samples with a “Normal” karyotype, for which cytogenetic evaluation had been conducted. This eliminated a small number of samples which were ambiguously annotated as normal karyotype, even though cytogenetic evaluation was indicated as ‘not done’. Of the 184 samples, non-APL had OS time and status, while 65 NKAML had OS information. For Tomasson et al., we required that either FAB subtype or karyotype information be available. Non-APL were then defined as the subset having non-M3 FAB and non-t(15;17) karyotype (or one of these when both annotations were not present). NKAML were selected as having “Normal” karyotype and non-M3 FAB (eliminating samples which were normal karyotype, but FAB M3). After this filtering, 137 non-APL samples had full OS and EFS data for survival analysis, while 70 NKAML had complete OS and EFS data. For Wouters et al., we similarly required that either FAB or karyotype be specified. Again, non-APL were defined as non-M3 and non-t(15;17), and NKAML as those with normal karyotype, and excluding M3. Following this, 219 non-APL had complete OS information, 99 NKAML had OS and EFS, and 85 NKAML had RFS. In multivariate analyses (see Results section, Table 5), the sample sizes indicated differ from those specified here because mutation and cytogenetic risk was not available in all cases.

Microarray Analysis.

We integrated data from 30 matched samples (15 pairs) of LSC-enriched and LPC-enriched samples from 11 patients with AML, and corresponding functionally defined mouse xenografts (Majeti R, et al. Proc Natl Acad Sci USA. 2009; 106(9):3396-3401; Ishikawa F, et al. Nat Biotechnol. 2007; 25(11):1315-1321; Hijikata A, et al. Bioinformatics. 2007; 23(21):2934-2941). The patients represented a diversity of subtypes and clinical outcomes (Table 9). Individual genes differentially expressed between paired LSC and LPC were identified using Significance Analysis of Microarrays (SAM) (Tusher V G, et al. Proc Natl Acad Sci USA. Apr. 24, 2001; 98(9):5116-5121), employing a paired metric (FDR<10%). We defined the ‘LSC signature’ as the first principal component of these genes in a given dataset across its samples. To identify biological themes distinguishing LSC from LPC, all genes were ranked by their geometric mean difference in expression between paired samples, and evaluated using Gene Set Enrichment Analysis (GSEA) (Mootha V, et al. Nature Genetics. 2003; 34(3):267-273). Raw microarray data were obtained as Affymetrix CEL files for four publicly available bulk AML gene expression studies 16-19 from NCBI GEO (GSE12417, n=163 normal-karyotype AML only, with OS outcomes; GSE10358, n=184, OS and EFS; GSE14468, n=527, OS, EFS and RFS) and NCl caArray (willm-00119, n=170 non-FAB M3, OS only). Details of patient characteristics, primary therapies, clinical responses, remission rates, and outcomes have been reported (see Table 8 above) (Metzeler K H, et al. An 86-probe-set gene-expression signature predicts survival in cytogenetically normal acute myeloid leukemia. Blood. 2008; 112(10):4193-4201; Tomasson M H, et al. Somatic mutations and germline sequence variants in the expressed tyrosine kinase genes of patients with de novo acute myeloid leukemia. Blood. 2008; 111(9):4797-4808; Wilson C S, et al. Gene expression profiling of adult acute myeloid leukemia identifies novel biologic clusters for risk classification and outcome prediction. Blood. Jul. 15, 2006; 108(2):685-696; Wouters B J, et al. Double CEBPA mutations, but not single CEBPA mutations, define a subgroup of acute myeloid leukemia with a distinctive gene expression profile that is uniquely associated with a favorable outcome. Blood. 2009; 113(13):3088-3091; Valk P J, et al. Prognostically useful gene-expression profiles in acute myeloid leukemia. N Engl J Med. 2004; 350(16):1617-1628). Ingenuity Pathways Analysis (IPA) was used to identify interaction networks of genes.

TABLE 9
Characteristics of patient samples used in defining the LSC signature genes. For Stanford
patients, age, gender, cytogenetic abnormalities, FAB subtype, FLT3-ITD status, time
from diagnosis to last followup, and status at last followup are reported. For the
independent dataset (Ishikawa et al.), only FAB subtype was available.
Stanford Samples

| | | | De Novo/ | | | | Time to last | Status at |
|---|---|---|---|---|---|---|---|---|
| Sample | Age | Gender | Relapsed | Cytogenetics | FAB | FLT3-ITD | followup (days) | last followup |
| SU001 | 59 | Female | Relapsed | Normal | M2 | Negative | 32 | DEAD |
| SU004 | 47 | Female | Relapsed | Normal | M5 | Positive | 74 | DEAD |
| SU006 | 51 | Female | De Nevo | n/a | M1 | Negative | 1196 | ALIVE |
| SU008 | 64 | Male | De Novo | Normal | M1 | Positive | 1102 | ALIVE |
| SU014 | 59 | Male | De Novo | Normal | n/a | Positive | 23 | ALIVE |
| SU031 | 31 | Female | De Novo | Complex | M4 | Negative | 708 | ALIVE |
| SU032 | 47 | Male | De Novo | Normal | M5 | Negative | 226 | ALIVE |

RIKEN primary AML samples

| Sample | Gender | De Novo/relapsed | FAB |
|---|---|---|---|
| Hs04 | Male | De Novo | M2 |
| Hs07 | Female | De Novo | M4 |
| Hs10 | Male | De Novo | M2 |
| Hs11 | Male | De Novo | M1 |

n/a = unknown

Microarray Renormalization and Analysis.

To compare data from different studies, all expression data were normalized from the raw CEL files. We used a custom CDF file to map Affymetrix probes to Refseq mRNA sequences (Dai, M. et al. Nucleic Acids Res. 2005. 33(20):e175). Array normalization was performed with the mass function of the affy package (v. 1.22.1) of Bioconductor version 2.4, under the R statistical programming environment (version 2.9.2). Arrays were scaled to have median intensity of 500. Differentially expressed genes between paired LSC and LPC samples were identified using the SAM package (v 1.26) in R.

Definition of LSC Signature.

The LSC signature was defined in specific datasets to be the first principal component of the genes up-regulated in LSC compared to LPC as determined by SAM. Principal components were computed using the prcomp function that is part of the base R installation. Genes up-regulated in LSC were chosen specifically under the following rationale. Consider a toy example of a tumor with 10 cells that each express a gene at the same intensity (call it 1). If one cell (10% of tumor) upregulates the gene 10-fold, the average expression across all cells becomes (1*10+9*1)/10=1.9. If one cell down regulates the gene 10-fold, average expression across all cells becomes (1*0.1+9*1)/10=0.91. Hence, expression changes of genes in a subpopulation are more readily detectable in bulk samples if they are more highly expressed than in the rest of the sample.

Definition of LSC Score.

To test associations between LSC-enriched genes and clinical outcomes, a retropective training-validation scheme was adopted. Raw microarray data were obtained for the 4 publicly available bulk AML gene expression studies with available clinical annotations; see Table 8 above. The LSC signature was calculated in the 163 NKAML samples of Metzeler et al. (training set). Weights from Principal Component Analysis derived in this set (Table 10) were then applied to independent datasets to define an LSC score for each patient sample. The expression values of the LSC genes in test cohorts were adjusted such that their NKAML samples had the same median expression as in the training cohort. This minimal standardization was intended to address the issue of variations in patient populations, sample collection, handling, processing, and microarray hybridization. To separate patients into LSC-high and LSC-low groups, the median LSC score in the training set was used and applied to the validation cohorts. The single exception was the Wilson et al. cohort. Since the array platform lacked probes for a number of LSC genes, the LSC score has a different range from the other cohorts (See Results section, Table 4). Nonetheless, the LSC score was continuously associated with survival in this dataset (Table 3). Hence, the high/low group for this dataset was defined based on the median LSC score within it.

TABLE 10
Weightings of genes in the LSC score. Tabulated are weights for individual genes over-
expressed in LSC-enriched populations that comprise the LSC score, with the latter representing
the weighted sum of the expression values of the genes for a given patient.

| | | | Weight |
|---|---|---|---|
| Probe | Description | Weight | (no CD34) |
| NM_001130687_at | GUCY1A3—Guanylate cyclase 1, soluble, | 0.598 | 0.623 |
| | alpha 3 | | |
| NM_006867_at | RBPMS—RNA binding protein with | 0.367 | 0.369 |
| | multiple splicing | | |
| NM_001145459_at | HOPX—HOP homeobox | 0.335 | 0.328 |
| NM_007351_at | MMRN1—Multimerin 1 | 0.254 | 0.269 |
| NM_001131005_at | MEF2C—Myocyte enhancer factor 2C | 0.253 | 0.261 |
| NM_001025109_at | CD34—CD34 molecule | 0.239 | — |
| XM_001716710_at | LOC100128550—Hypothetical protein | 0.162 | 0.170 |
| | LOC100128550 | | |
| NM_001142472_at | FAIM3—Fas apoptotic inhibitory molecule 3 | 0.159 | 0.154 |
| NM_004666_at | VNN1—Vanin 1 | 0.146 | 0.196 |
| NM_015660_at | GIMAP2—GTPase, IMAP family member 2 | 0.146 | 0.148 |
| NM_001077484_at | SLC38A1—Solute carrier family 38, | 0.137 | 0.130 |
| | member 1 | | |
| NM_002341_at | LIB—Lymphotoxin beta (TNF superfamily, | 0.137 | 0.139 |
| | member 3) | | |
| NM_015559_at | SETBP1—SET binding protein 1 | 0.117 | 0.093 |
| NM_002126_at | HLF—Hepatic leukemia factor | 0.108 | 0.113 |
| NM_024711_at | GIMAP6—GTPase, IMAP family member 6 | 0.091 | 0.094 |
| NM_017439_at | PION—Pigeon homolog (Drosophila) | 0.083 | 0.094 |
| XM_001126245_at | LOC727893—Similar to phosphodiesterase | 0.077 | 0.080 |
| | 4D interacting protein (myomegalin) | | |
| NM_052913_at | TMEM200A—Transmembrane protein 200A | 0.051 | 0.041 |
| NM_153236_at | GIMAP7—GTPase, IMAP family member 7 | 0.044 | 0.018 |
| NM_024768_at | CCDC48—Coiled-coil domain containing 48 | 0.041 | 0.034 |
| NM_032295_at | SLC37A3—Solute carrier family 37, | 0.031 | 0.016 |
| | member 3 | | |
| NM_014181_at | HSPC159—Galectin-related protein | 0.013 | 0.008 |
| NM_015002_at | FBXO21—F-box protein 21 | 0.011 | 0.004 |
| NM_032090_at | PCDHGC3—Protocadherin gamma subfamily C, 3 | −0.01 | −0.025 |
| NM_001003927_at | EVI2A—Ecotropic viral integration site 2A | −0.019 | −0.006 |
| NM_001005463_at | EBF3—Early B-cell factor 3 | −0.019 | −0.031 |
| NM_001165_at | BIRC3—Baculoviral IAP repeat-containing 3 | −0.025 | −0.023 |
| NM_016217_at | HECA—Headcase homolog (Drosophila) | −0.053 | −0.065 |
| NM_001018009_at | SH3BP5—SH3-domain binding protein 5 | −0.055 | −0.060 |
| | (BTK-associated) | | |
| NM_000392_at | ABCC2—ATP-binding cassette, sub- | −0.082 | −0.085 |
| | family C (CFTR/MRP), member 2 | | |
| NM_000201_at | ICAM1—Intercellular adhesion molecule 1 | −0.099 | −0.107 |

Statistical Analysis.

An LSC score was defined in a training set of 163 NKAML samples to be the first principal component of expression of LSC-enriched genes in that cohort (Metzeler K H, et al. An 86-probe-set gene-expression signature predicts survival in cytogenetically normal acute myeloid leukemia. Blood. 2008; 112(10):4193-4201). Gene weightings defined in the training cohort were applied to three independent test cohorts to derive a corresponding LSC score for each sample. The median LSC score in the training set was used to partition patients in all cohorts into high- and low-score groups.

The LSC score was tested for associates with survival outcomes as a continuous variable using Cox proportional hazards regression (log-likelihood test), and as a dichotomous stratification (high vs low LSC score) using Kaplan-Meier analysis (log-rank test) using R version 2.11 with survival package 2.35 (R project for Statistical Computing [found on the world wide web at address www.R-project.org]). For relapse-free survival (RFS), we included only patients who had first achieved clinical remission from disease (Dohner H, et al. Diagnosis and management of acute myeloid leukemia in adults: recommendations from an international expert panel, on behalf of the European LeukemiaNet. Blood. 2010; 115(3):453-474). Association of the LSC score to AML subgroups was assessed by ANOVA. As assignments of patients to cytogenetic risk groups were inconsistent between different clinical groups, we compared risk across datasets in uniform fashion, by applying the refined Medical Research Council (MRC) risk scheme (favorable, intermediate, adverse) based on karyotype (Grimwade D, Hills R K. Independent prognostic factors for AML outcome. Hematology Am Soc Hematol Educ Program. 2009:385-395).

Associations of the LSC signature or score between different subgroups were assessed using default R functions for t-test, Wilcoxon Rank Sum test. Normality of distributions compared by t-test were evaluated by normal-quantile plots. Different karyotypic groups differed significantly in their sample size, and in the variance of the LSC signature within them. To account for this, the Games-Howell post-hoc test (which does not assume equal variances or sample sizes) was used to determine statistical significance of LSC signature differences between karyotypes. The latter analysis was carried out in SPSS 12 (IBM Inc.).

Independence from Tissue of Origin.

In the AML studies analyzed for outcome associations, bulk leukemic specimens had been obtained from either bone marrow aspirates (BM) or peripheral blood (PB) of patients with AML. Accordingly, to test if the LSC score was independent of AML tissue origin, it was first evaluated in paired gene expression data of 5 AML samples obtained from the BM and PB of the same patient 2. Unsupervised clustering showed that AML samples from the same patient invariably grouped together with bootstrap-derived probabilities >98% (FIG. 11), indicating that the signature is expressed similarly in AML cells obtained from either the BM or PB.

Survival Analysis.

The analyses of survival were carried out in R using the Design (v 2.2) and survival (v 2.35) packages. In multivariate Cox analyses, the LSC score and patient age (in years) were modeled as continuous variables. FLT3-ITD and NPM1c were designated as ‘0’ for wild-type and ‘1’ for mutated. Cytogenetic risk groups (per Revised MRC Risk Group Criteria) were coded as 1=“Favorable”, 2=“Intermediate”, and 3=“Adverse”. For associations with survival of continuous variables (e.g. LSC score) we report the log-likelihood p-values. For discrete variables (e.g. high/low-LSC groups) the log-rank p-values determined from Kaplan-Meier analysis are reported.

Analysis of area under curve (AUC) for the Receiver Operating Characteristic (ROC) curve was conducted using the survival ROC package in R, allowing for time-dependent ROC curve estimation with censored data. Since in all of the survival analyses, few events occurred after 2 years (see Kaplan-Meier curves), we compared the ability of models to predict OS at this time point. The ROC curve plots the true-positive vs. false-positive predictions, thus higher AUC indicates better model performance (with AUC=0.5 indicating no better than random). LSC scores and groups (LSC-high or LSC-low, defined above), based on weightings derived in the training cohort (Metzeler). For NKAML, multivariate models incorporating age, FLT3, NPM1, LSC score were built in Metzeler, and the same parameters were then applied to predict the combined score of these variables in the NKAML samples from the other cohorts. For karyotypically abnormal AML excluding APL, data from Tomasson et al was used for training, with the parameters derived in that set applied to the other two cohorts of non-APL patients. ROC curves for OS at 2 years were constructed for a) LSC score alone in NKAML and non-APL AML, b) the multivariate combination of age, FLT3, NPM1 status for NKAML, and c) Age, FLT3, NPM1 status and cytogenetic risk for non-APL. Comparison ROC curves were then built for (b) and (c) combined with the dichotomous LSC group (high/low=1/0). The ability of these models to predict OS at 2 years was compared by the AUC of their ROC curves (see Results section, Table 7).

Robustness of LSC Score: Cross-Validation in Training Set.

For assessing robustness of the LSC score, sub-sampling and cross-validation were employed. The NKAML training set (Metzeler et al) was split randomly into two equal subsets. The first was used to define gene weights for an LSC score, which was then assessed for its ability to predict OS (p-value, HR and 95% CIs of HR) in the second subset. This procedure was repeated 1000 times.

Analysis of Random Gene Sets.

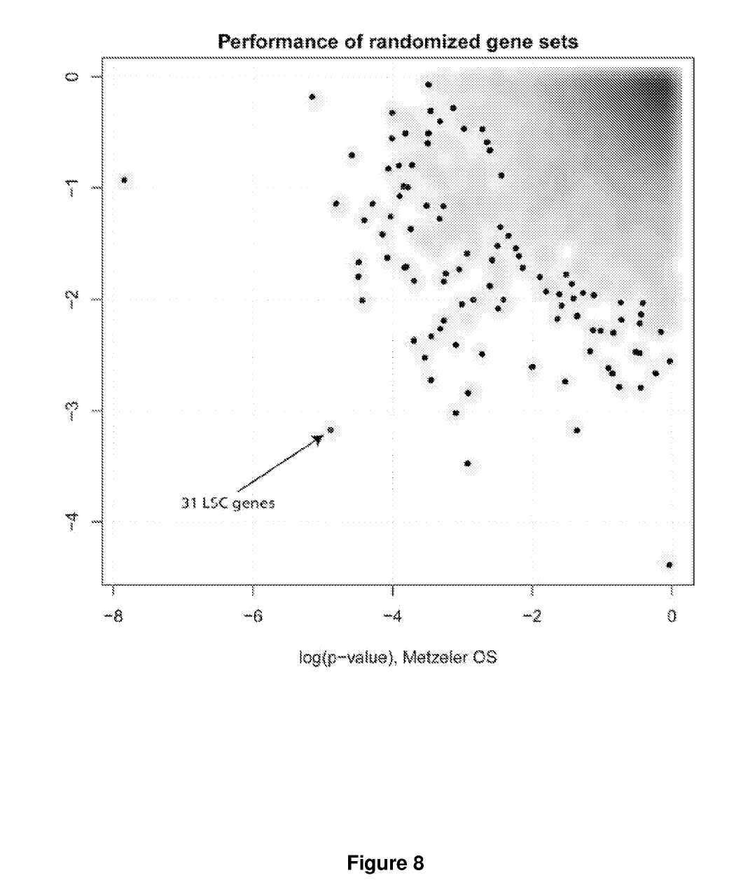

In order to determine how likely the association of the LSC score with survival outcomes was to occur by chance, its performance was compared to random sets of genes of the same size (31 genes). Here, groups of 31 genes were randomly selected in the training set, and their “score” was defined in the same way as the LSC score (first principal component), and this was assessed for its ability to predict OS in the training set. Analogously to cross-validation of the LSC score, the gene weightings from the training set were applied to the test cohorts, and association with OS tested. This procedure was repeated 10000 times, revealing the group of 31-genes defining the LSC score as exceptional. Results are shown in FIG. 8.

### Example 2

Prognostic models for prediction of overall, event-free, and/or relapse-free survival in acute myeloid leukemia (AML) are provided that are based upon the expression of three genes (HOPX, GUCY1A3, and CCL5) in various predictive combinations. These genes are differentially expressed between leukemic stem cells (LSC) and non-tumor initiating cells (see, e.g., FIG. 1), and comprise a measure of LSC activity in AML.

The models are generally applicable to expression data obtained from any convenient methodology, e.g. microarray analysis, polymerase chain reaction (PCR), transcriptome sequencing, and the like. The prognostic power of this diagnostic test is applicable to both normal karyotype AML (NKAML) and AML with cytogenetic abnormalities. The predictor is prognostic of outcomes independently of other clinical covariates including age, cytogenetic risk, FLT3-ITD, NPM1, and CEBPa mutation status. Several alternative forms of the predictor are also described, for use as a continuous score that can be used for classification of patient risk group, and also as a set of expression thresholds that can be used to construct an integer score for a given patient that describes their relative risk.

### Description of Models

Expression of the 3 reporter genes (HOPX, GUCY1A3, and CCL5) is determined by microarray, PCR, or other methods. After transformation of expression measures to log-space, expression values of each gene in a sample are normalized relative to the expression of one of several possible control housekeeping genes, for example, ABL1, GAPDH, or PGK1. Here we demonstrate the utility of ABL1; performance using GAPDH and PGK1 is similar. Following normalization of each of these expression values relative to the control gene expression, a continuous score may be ascribed for the patient in one of the following three models:

Model 1, which relies upon the expression values obtained for HOPX and GUCY1A3. In this model, the LSC signature score is arrived at as follows:

score=(0.18)(normalized HOPX value)+(0.10)(normalized GUCY1A3 value).

Model 2, which relies upon the expression values obtained for HOPX and CCL5. In this model, the LSC signature score is arrived at as follows:

score=(0.24)(normalized HOPX value)−(0.11)(normalized CCL5 value).

Model 3, which relies upon the expression values obtained for HOPX and GUCY1A3 and CCL5. In this model, the LSC signature score is arrived at as follows:

score=(0.20)(normalized HOPX value)+(0.09)(normalized GUCY1A3 value)−(0.21)(normalized CCL5 value).

### Results

Performance of Prognosis Models Based Upon HOPX, GUXY1A3 and CCL5 Gene Expression Profiles.

The performance of Models 1, 2 and 3 for predicting OS, EFS, and RFS in training set (Metzeler) and test sets (Metzeler2, Tomasson, Wouters) in both normal karyotype AML (NKAML) patients and across all AML patients (excluding acute promyelocytic leukemia, APL) patients is shown in Table 11. Hazard ratios (HR) with 95% confidence intervals (95% CI) and p-values for the score as a continuous predictor are given.

TABLE 11
Performance of two and three gene models in training and validation sets. Gene expression for combinations
of HOPX, GUCY1A3, and CCL5 were constructed in the training set (Metzeler), with derived weights shown
in the “Model” column. These weights were applied to the indicated test datasets to derive a score
for each patient, which was tested for association with OS, EFS, and RFS in normal karyotype AML, and across
all non-APL AMLs. Hazard ratios are shown with 95% CIs, along with log-likelihood test p-values.

| | | Train | | NKAML test | | | | |
|---|---|---|---|---|---|---|---|---|
| | | Metzeler | | Metzeler2 | | Wouters | | Tomasson |
| | Model | HR (95% CI) | p | HR (95% CI) | p | HR (95% CI) | p | HR (95% CI) |
| OS | 0.18*HOPX + | 2.7 (1.8-4.1) | 4e−7 | 2.2 (1.2-3.8) | 5e−3 | 2.5 (1.4-4.3) | 1e−3 | 2.3 (1.5-3.5) |
| | 0.10*GUCY1A3 | | | | | | | |
| OS | 0.24*HOPX − | 2.7 (1.9-4.0) | 3e−7 | 2.8 (1.5-5.0) | 6e−4 | 2.4 (1.4-4.0) | 2e−3 | 1.9 (1.3-2.8) |
| | 0.22*CCL5 | | | | | | | |
| OS | 0.20*HOPX + | 2.7 (1.9-3.9) | 2e−8 | 2.6 (1.5-4.4) | 3e−4 | 2.7 (1.6-4.4) | 1e−4 | 1.8 (1.2-2.6) |
| | 0.09*GUCY1A3 − | | | | | | | |
| | 0.21*CCL5 | | | | | | | |
| EFS | 0.18*HOPX + | | | 2.9 (1.7-5.1) | 7e−5 | 2.3 (1.4-3.9) | 1e−3 | 2.4 (1.5-3.7) |
| | 0.10*GUCY1A3 | | | | | | | |
| EFS | 0.24*HOPX − | | | 3.7 (2.1-6.7) | 3e−6 | 2.1 (1.3-3.4) | 4e−3 | 2.0 (1.3-3.0) |
| | 0.22*CCL5 | | | | | | | |
| EFS | 0.20*HOPX + | | | 3.1 (1.9-5.3) | 5e−6 | 2.5 (1.6-4.0) | 1e−4 | 1.9 (1.3-2.8) |
| | 0.09*GUCY1A3 − | | | | | | | |
| | 0.21*CCL5 | | | | | | | |
| RFS | 0.18*HOPX + | | | 3.3 (1.4-7.9) | 4e−3 | 1.8 (0.9-3.5) | 0.07 | |
| | 0.10*GUCY1A3 | | | | | | | |
| RFS | 0.24*HOPX − | | | 3.9 (1.6-9.6) | 2e−3 | 1.7 (0.9-3.3) | 0.11 | |
| | 0.22*CCL5 | | | | | | | |
| RFS | 0.20*HOPX + | | | 3.2 (1.4-7.0) | 3e−3 | 2.0 (1.1-3.7) | 0.02 | |
| | 0.09*GUCY1A3 − | | | | | | | |
| | 0.21*CCL5 | | | | | | | |

| | | NKAML test | non-APL test | | | |
|---|---|---|---|---|---|---|
| | | Tomasson | Wouters | | Tomasson | |
| | Model | p | HR (95% CI) | p | HR (95% CI) | p |
| OS | 0.18*HOPX + | 3e−4 | 1.8 (1.2-2.5) | 2e−3 | 2.0 (1.4-2.7) | 6e−5 |
| | 0.10*GUCY1A3 | | | | | |
| OS | 0.24*HOPX − | 2e−3 | 1.7 (1.2-2.4) | 1e−3 | 1.7 (1.3-2.3) | 2e−4 |
| | 0.22*CCL5 | | | | | |
| OS | 0.20*HOPX + | 2e−3 | 1.6 (1.2-2.2) | 2e−3 | 1.7 (1.3-2.3) | 1e−4 |
| | 0.09*GUCY1A3 − | | | | | |
| | 0.21*CCL5 | | | | | |
| EFS | 0.18*HOPX + | 2e−4 | 1.8 (1.3-2.6) | 6e−4 | 1.4 (1.0-2.1) | 0.04 |
| | 0.10*GUCY1A3 | | | | | |
| EFS | 0.24*HOPX − | 9e−4 | 1.7 (1.3-2.3) | 7e−4 | 1.3 (1.0-1.8) | 0.07 |
| | 0.22*CCL5 | | | | | |
| EFS | 0.20*HOPX + | 1e−3 | 1.7 (1.3-2.2) | 4e−4 | 1.3 (1.0-1.8) | 0.06 |
| | 0.09*GUCY1A3 − | | | | | |
| | 0.21*CCL5 | | | | | |
| RFS | 0.18*HOPX + | | 1.3 (0.8-2.0) | 0.24 | | |
| | 0.10*GUCY1A3 | | | | | |
| RFS | 0.24*HOPX − | | 1.3 (0.9-2.0) | 0.17 | | |
| | 0.22*CCL5 | | | | | |
| RFS | 0.20*HOPX + | | 1.3 (0.9-1.9) | 0.19 | | |
| | 0.09*GUCY1A3 − | | | | | |
| | 0.21*CCL5 | | | | | |

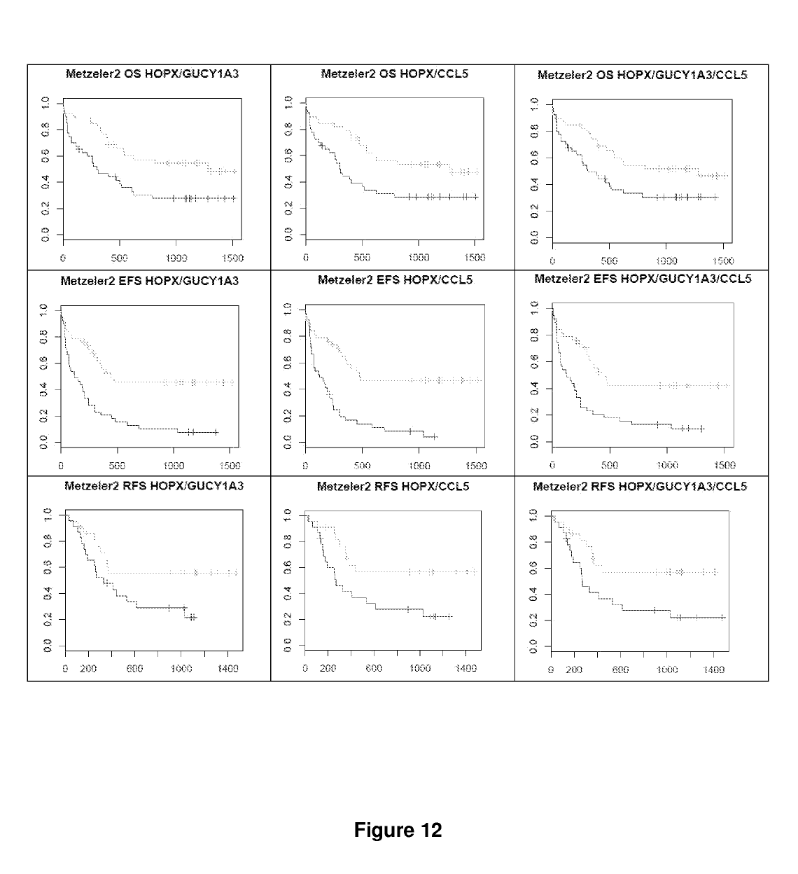

Based on the scores in Table 11, patients can be ascribed to high- or low-risk categories depending on whether their score is higher or lower than the median score across a cohort. Kaplan-Meier curves are shown in FIG. 12 for OS, EFS, and RFS for each model in the Metzeler2 validation dataset. Horizontal axis shows survival time in days; vertical shows probability of event occurring. Other groupings, besides median stratification, can be generated by selection of specific score thresholds in the training set, and cross-validated against the test sets (Table 11 and FIG. 12).

Performance of Models in Combination with Other Clinical Covariates.

In AML, age, cytogenetic risk group, and NPM1/FLT3 mutation status have been described as contributing to patient risk. Table 12 and Table 13 demonstrate that LSC score adds to these by multivariate Cox regression. We show here the 3-gene model with genes normalized to ABL1. Scoring is based on thresholds for individual genes (AIM procedure)

An alternative approach to applying the three aforementioned genes to AML prognosis is by specifying thresholds in each. Every patient receives an initial risk score of zero. If Gene 1 exceeds (or is less than) a specific level of expression, the patient receives a +1 contribution to score, otherwise 0.

TABLE 12
Multivariate performance of the three gene model derived in the training set (Metzeler) after genes were normalized
to ABL1 to simulate the effect in PCR of normalizing to a housekeeping gene (for which ABL1 is a potential
candidate). Shown are the performances within NKAML subsets of the data. The multivariate model combines the
3 gene LSC score with age, and FLT3/NPM1 mutation status. Shown are the HRs and p values for each variable
within the multivariate model, together the performance of the “overall” model that combines them.

| | | Metzeler train | | Metzeler2 test | | Wouters NKAML | | Tomasson NKAML | |
|---|---|---|---|---|---|---|---|---|---|
| OS | 3-gene ABL1 score | 2.0 (1.4-3.0) | 4e−4 | 1.9 (1.1-3.4) | 0.02 | 2.6 (1.4-4.6) | 2e−3 | 2.1 (1.3-3.2) | 1e−3 |
| | Age | 1.02 (1.00-1.04) | 5e−3 | 1.03 (1.0-1.06) | 0.02 | 1.02 (1.0-1.04) | 0.14 | 1.01 (0.99-1.03) | 0.24 |
| | FLT3 | 2.1 (1.4-3.3) | 7e−4 | 1.5 (0.8-3.0) | 0.20 | 1.8 (1.1-3.1) | 0.02 | 2.7 (1.3-5.3) | 5e−3 |
| | NPM1 | 0.9 (0.6-1.4) | 0.63 | 0.6 (0.3-1.3) | 0.21 | 0.8 (0.5-1.4) | 0.45 | 1.7 (0.9-3.3) | 0.13 |
| | Overall | | 1e−9 | | 4e−4 | | 3e−4 | | 8e−5 |
| EFS | 3-gene ABL1 score | | | 2.4 (1.4-4.1) | 2e−3 | 2.6 (1.5-4.4) | 5e−4 | 2.2 (1.4-3.4) | 6e−4 |
| | Age | | | 1.02 (1.0-1.04) | 0.09 | 1.0 (0.98-1.03) | 0.54 | 1.0 (0.99-1.03) | 0.52 |
| | FLT3 | | | 1.3 (0.7-2.5) | 0.36 | 2.0 (1.2-3.3) | 7e−3 | 2.7 (1.3-5.6) | 8e−3 |
| | NPM1 | | | 0.6 (0.3-1.1) | 0.08 | 1.0 (0.6-1.8) | 0.95 | 1.5 (0.7-2.9) | 0.27 |
| | Overall | | | | 3e−5 | | 8e−5 | | |
| RFS | 3-gene ABL1 score | | | 2.4 (1.0-5.6) | 0.04 | 2.0 (1.0-4.0) | 0.05 | | |
| | Age | | | 1.02 (1.0-1.06) | 0.17 | 1.0 (0.97-1.03) | 0.99 | | |
| | FLT3 | | | 1.5 (0.6-3.7) | 0.39 | 2.6 (1.4-5.1) | 4e−3 | | |
| | NPM1 | | | 0.6 (0.3-1.5) | 0.30 | 1.1 (0.5-2.2) | 0.89 | | |
| | Overall | | | | 0.02 | | 4e−3 | | |

TABLE 13
Multivariate performance of the three gene model derived in the
training set (Metzeler) after genes were normalized to ABL1 to simulate
the effect in PCR of normalizing to a housekeeping gene (for which
ABL1 is a potential candidate) as described for Table 12, but including
cytogenetic risk into the model (for the two datasets that contain
samples with cytogenetic abnormalities).

| | | Wouters | | Tomasson | |
|---|---|---|---|---|---|
| | | NKAML | | NKAML | |
| OS | 3-gene | 1.3 (1.0-1.8) | 0.07 | 1.7 (1.2-2.3) | 9e−4 |
| | ABL1 score | | | | |
| | Age | 1.01 (1.0-1.03) | 0.03 | 1.02 (1.0-1.04) | 5e−3 |
| | FLT3 | 2.0 (1.3-2.9) | 7e−4 | 1.8 (1.1-3.0) | 0.03 |
| | NPM1 | 0.6 (0.4-1.0) | 0.04 | 1.7 (1.0-2.7) | 0.04 |
| | Cyto risk | 2.0 (1.5-2.6) | 2e−6 | 1.9 (1-3-2.9) | 2e−3 |
| | | | 3e−9 | | 2e−8 |
| EFS | 3-gene | 1.4 (1.0-1.8) | 0.03 | 1.4 (1.0-2.0) | 0.03 |
| | ABL1 score | | | | |
| | Age | 1.0 (0.99-1.02) | 0.67 | 1.0 (0.99-1.02) | 0.84 |
| | FLT3 | 1.8 (1.3-2.7) | 1e−3 | 1.8 (1.0-3.0) | 0.06 |
| | NPM1 | 0.7 (0.5-1.0) | 0.06 | 1.7 (1.0-2.9) | 0.04 |
| | Cyto risk | 1.9 (1.4-2.5) | 7e−6 | 1.4 (0.9-2.1) | 0.19 |
| | | | 3e−8 | | 0.01 |
| RFS | 3-gene | 1.1 (0.7-1.5) | 0.73 | | |
| | ABL1 score | | | | |
| | Age | 1.0 (0.99-1.03) | 0.34 | | |
| | FLT3 | 2.0 (1.2-3.2) | 7e−3 | | |
| | NPM1 | 0.7 (0.4-1.1) | 0.12 | | |
| | Cyto risk | 2.1 (1.4-3.0) | 1e−4 | | |
| | | | 2e−4 | | |

### Example 3

Prognostic models for prediction of overall, event-free, and/or relapse-free survival in acute myeloid leukemia (AML) are provided that are based upon the expression of three genes (HOPX, GUCY1A3, and IL2RA). These genes are differentially expressed between leukemic stem cells (LSC) and non-tumor initiating cells, and comprise a measure of LSC activity in AML.

We identified the core set of LSC-related genes that carry most prognostic weight, and which can be combined into a prognostic assay using qt-PCR which is commonly used in clinical practice. Four existing gene expression cohorts were combined into one set of 1042 patient samples. 773 samples had available outcome data. These 773 were split into ⅔ training and ⅓ test sets. The prognostic power of each candidate gene associated with LSC (our ˜52 together with additional genes mentioned i.e. IL2RA, MSI2) was evaluated by univariate Cox regression in the training set. This analysis was performed by randomly selecting ½ of the training set, to evaluate the robustness. Genes were selected that were prognostically significant (p<0.05) in at least 500 of 1000 random samplings. We then evaluated all possible models combining 2 or more of these genes into one LSC score. This step was again performed using Cox regression on 1000 random splits of the training set. From this, the most robust and prognostic set of genes was found to be the combination of HOPX, GUCY1A3, and IL2RA (CD25). In the training set, the optimal LSC score was

score=4*HOPX+3*IL2RA+3*GUCY1A3

However, a score with equal weighting of the genes was nearly as prognostic and robust, as was a score constructed from the pair HOPX and IL2RA. A score constructed from HOPX alone also had strong performance.

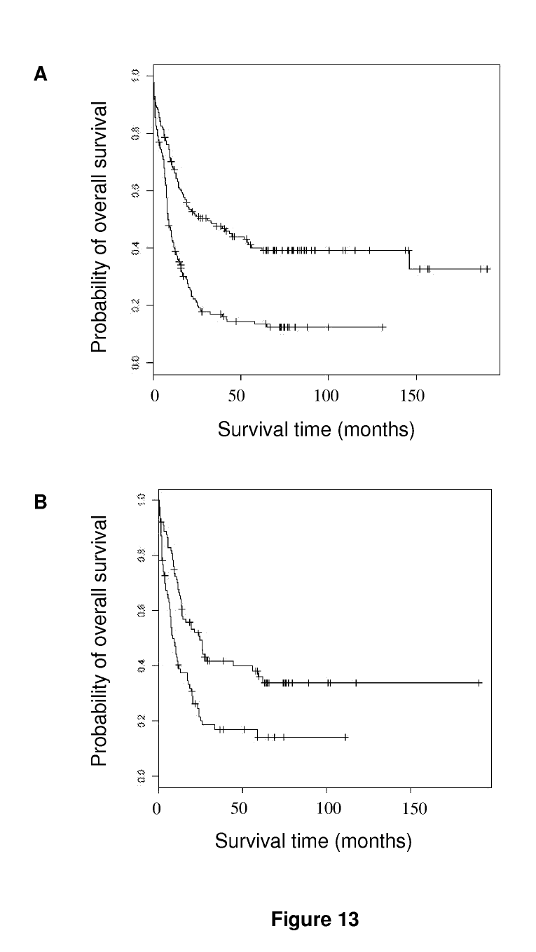

The score was evaluated in the test set (the ⅓ left out above). The Kaplan-Meier survival curves for overall survival when patients were split into high/low score (defined as being above or below the median) are shown in FIG. 13 in the intermediate cytogenetic risk group (which comprises ⅔ of all AML patients). The statistical analysis of 3-gene LSC score for univariate association with overall survival in cytogenetically intermediate risk patients is shown in Table 14, and the multivariate analysis with other prognostic variables across all patients is shown in Table 15.

TABLE 14
Statistical analysis of 3-gene LSC score for univariate association
with overall survival in cytogenetically intermediate risk patients.

| | Training set (n = 344) | | Test set (n = 165) | |
|---|---|---|---|---|
| | Hazard ratio | | Hazard ratio | |
| | (95% CI) | p | (95% CI) | p |
| LSC score continuous | 1.8 (1.6-2.2) | *10⁻¹² | 1.5 (1.2-2.0) | .0005 |
| LSC score high vs low | 2.3 (1.8-3.0) | *10⁻¹¹ | 2.0 (1.4-3.0) | .0001 |

TABLE 15
Multivariate analysis with other prognostic
variables across all patients.

| | Training set (n = 481) | | Test set (n = 238) | |
|---|---|---|---|---|
| | Hazard ratio | | Hazard ratio | |
| | (95% CI) | p | (95% CI) | p |
| LSC score | 1.4 (1.2-1.6) | e−5* | 1.4 (1.1-1.8) | .005* |
| continuous | | | | |
| Age | 1.02 (1.01-1.03) | 6e−10 | 1.02 (1.01-1.04) | 7e−5 |
| FLT3_ITD | 1.9 (1.4-2.4) | 1e−6 | 1.7 (1.2-2.5) | 0.005 |
| NPM1 | 0.8 (0.6-1.0) | 0.13 | 1.0 (0.7-1.5) | 0.89 |
| CytoRisk | 2.1 (1.7-2.7) | 1e−9 | 1.7 (1.2-2.4) | 0.002 |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of providing a prognosis for a patient with a hematological malignancy, the method comprising:
a. obtaining a leukemia stem cell (LSC) expression representation for a hematologic sample from said patient, wherein said LSC expression representation represents the expression level of one or more LSC genes selected from the group consisting of CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T; and
b. employing the LSC expression representation to provide the prognosis for said patient.

2. The method according to claim 1, wherein the LSC expression representation represents measurements of the expression levels of at least the genes HOPX and GUCY1A3.

3. The method according to claim 1, wherein the LSC expression representation further represents measurements of the expression level of the IL2RA gene.

4. The method according to claim 3, wherein the LSC expression representation represents measurements of the expression levels of at least the genes HOPX and IL2RA.

5. The method according to claim 3, wherein the LSC expression representation represents measurements of the expression levels of at least the genes HOPX, GUCY1A3 and IL2RA.

6. The method according to claim 1, wherein the LSC expression representation represents measurements of the expression levels of at least the genes CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, IL2RA, PCDHGC3, PION, RBPMS, SETBP1, SH3BP5, ABCC2, FBXO21, HECA, HLF, LOC100128550, LTB, MEF2C, SLC37A3, and TMEM200A.

7. The method according to claim 1, wherein said hematologic sample is a peripheral blood sample or a bone marrow sample.

8. The method according to claim 7, wherein said hematologic sample comprises an enriched population of leukemia stem cells (LSC).

9. The method according to claim 1, wherein the LSC expression representation is an LSC expression profile of the normalized expression level of each of said one or more genes.

10. The method according to claim 1, wherein the LSC expression representation is an LSC score, wherein an LSC score is calculated from the weighted normalized expression level of each of said one or more genes in a reference dataset.

11. The method according to claim 1, wherein employing the LSC expression representation comprises comparing the LSC expression representation to the LSC expression representation of one or more reference samples.

12. The method according to claim 1, wherein the hematological malignancy is a lymphoma, a leukemia, or a multiple myeloma.

13. The method according to claim 12, wherein the leukemia is acute myelogenous leukemia (AML).

14. The method according to claim 1, wherein the disease prognosis is a prognosis of overall survival (OS), relapse-free survival (RFS) and/or event-free survival (EFS).

15. A kit for use in providing a prognosis for a patient with a hematological malignancy, the kit comprising:
reagents to obtain an LSC expression representation from a hematologic sample from a patient; and
an LSC expression representation reference.

16. A method of screening a candidate agent for the ability to inhibit a hematological malignancy, the method comprising:
a. contacting a hematologic sample with a candidate agent;
b. obtaining an LSC expression representation from the contacted hematologic sample;
c. comparing said LSC expression representation from the contacted hematologic sample to the LSC expression representation from a hematologic sample that has not be contacted with the agent, and
d. employing the result of the comparison to determine the ability of the candidate agent to inhibit a hematological malignancy.

17. The method according to claim 16, wherein the contacting step occurs in vitro.

18. The method according to claim 16, wherein the contacting step occurs in vivo.

19. The method according to claim 16, wherein the LSC expression representation represents the expression level in the hematologic sample of one or more genes selected from the group consisting of CCDC48, FAIM3, GIMAP2, GIMAP7, HSPC159, LOC727893, MMRN1, SLC38A1, VNN1, BIRC3, CD34, EBF3, EVI2A, GIMAP6, GUCY1A3, HOPX, ICAM1, PCDHGC3, PION, RBPMS, SETBP1, SH3BP5, ABCC2, FBXO21, HECA, HLF, LOC100128550, LTB, MEF2C, SLC37A3, TMEM200A, CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T.

20. The method according to claim 19, wherein the LSC expression representation further represents the measurement of the expression level of the IL2RA gene.

21. The method according to claim 20, wherein a decrease in the LSC expression representation of one or more genes selected from the group consisting of CCDC48, FAIM3, GIMAP2, GIMAP7, HSPC159, IL2RA, LOC727893, MMRN1, SLC38A1, VNN1, BIRC3, CD34, EBF3, EVI2A, GIMAP6, GUCY1A3, HOPX, ICAM1, PCDHGC3, PION, RBPMS, SETBP1, SH3BP5, ABCC2, FBXO21, HECA, HLF, LOC100128550, LTB, MEF2C, SLC37A3, and TMEM200A indicates that the candidate agent inhibits the hematological malignancy.

22. The method according to claim 19, wherein an increase in the LSC expression representation of one or more genes selected from the group consisting of CD38, CSTA, DDX53, RNASE2, RNASE3, NM_{—}001146015, ANLN, C13orf3, CCL5, CCNA1, CLC, CPA3, DLGAP5, IL1F8, KIAA0101, MND1, MS4A3, OLFM4, STAR, ZWINT, and UBE2T indicates that the candidate agent inhibits the hematological malignancy.
